(19) United States
(12) Patent Application Publication
(10) Pub. No.: US 2019/0134101 A1
(43) Pub. Date: May 9, 2019

(54) GENETICALLY MODIFIED HUMAN UMBILICAL CORD PERIVASCULAR CELLS FOR WOUND HEALING

(71) Applicant: Tissue Regeneration Therapeutics Inc., Toronto (CA)

(72) Inventors: Lorena Ruth Braid, Medicine Hat (CA); John E. Davies, Toronto (CA)

(21) Appl. No.: 16/309,741

(22) PCT Filed: Jun. 15, 2017
(86) PCT No.: PCT/CA2017/000149

§ 371 Date: Dec. 13, 2018

Related U.S. Application Data
(60) Provisional application No. 62/350,641, filed on Jun. 15, 2016.

(51) Int. Cl. A61K 35/51; A61K 45/06; A61K 35/28; A61P 17/02; C07K 14/78; C12N 5/0775; C07K 14/54; C07K 14/475

(52) U.S. Cl. CPC A61K 35/51; A61K 45/06; A61K 35/28; C07K 14/475; C07K 14/78; C12N 5/0665; C07K 14/5428; A61P 17/02

(57) ABSTRACT

The invention features compositions including genetically modified human umbilical cord perivascular cells (HUCPVCs), medium conditioned by HUCPVCs (e.g., genetically modified HUCPVCs), the soluble fraction of medium conditioned by HUCPVCs (e.g., genetically modified HUCPVCs), and pharmaceutical compositions thereof, and methods of use thereof for treatment of wounds.

### BACKGROUND OF THE INVENTION

Wound healing is a multifaceted process orchestrated by numerous cell types and a complex interplay of signals emanating from the damaged cells and mediators of the immune response. For example, injury to the skin initiates a cascade of events including clot formation, cell migration, extracellular matrix synthesis and deposition, and finally, dermal and epidermal reconstitution and re-modeling. Tissue disruption in humans does not result in tissue regeneration, but in a rapid repair process leading to a fibrotic scar. Massive and chronic skin wounds commonly produce excessive scars which lack both form and function. Pathological scarring produces non-functional tissue at the wound site, leading to skin dysfunction, deformities, and restricted mobility, and may lead to disability and psychological trauma. The financial burden of cosmetic surgery, as well as physical and psychiatric rehabilitation to treat patients with pathological scarring, is staggering.

For these reasons, current research has focused on regeneration. For example, with regards to skin, the aim is to re-direct the physiological wound healing response from the deposition of non-functional tissue (scars) to a process that regenerates functional skin structure, including all epidermal appendages including hair follicles and sweat glands. Genes that encode factors with anti-scarring effects typically have roles in counteracting inflammation. Rapid resolution of the inflammatory phase accelerates wound closure and minimizes the natural over-production of matrix molecules used to contract the wound, which later gives rise to scar tissue.

Application of mesenchymal stem cells (MSCs) to wounds, such as excision and burn wounds, has been shown to improve the rate and integrity of the natural healing process. Mounting evidence suggests that transplanted MSCs elicit a paracrine response that recruits endogenous stem cells to the site of injury, and also attenuates the inflammatory response of the host. Human MSCs are immune-privileged, and therefore can typically be transplanted between individuals without rejection. However, current MSC-based therapies do not produce seamless skin regeneration or eliminate scarring; suggesting that the innate ability of MSCs to improve wound healing is limited.

Therefore, there remains a need for improved compositions and methods for treating wounds.

### SUMMARY OF THE INVENTION

The invention features human umbilical cord perivascular cells (HUCPVCs) which have been genetically modified to express a wound healing agent; medium conditioned by HUCPVCs (e.g., genetically modified HUCPVCs); compositions that include the soluble fraction of medium conditioned by HUCPVCs (e.g., genetically modified HUCPVCs); and pharmaceutical compositions that include genetically modified HUCPVCs, medium conditioned by HUCPVCs (e.g., genetically modified HUCPVCs), and/or the soluble fraction of such medium. Also featured are methods of using these compositions for the treatment of wounds.

In a first aspect, the invention features a human umbilical cord perivascular cell (HUCPVC) which has been genetically modified to express a wound healing agent selected from a non-antibody anti-fibrotic factor, a non-antibody anti-inflammatory factor, a stem cell recruitment factor, and an extracellular matrix factor.

In several embodiments of the first aspect of the invention, the non-antibody anti-fibrotic factor is a transforming growth factor (TGF)-β antagonist. In some embodiments, the TGF-β antagonist is decorin.

In several embodiments of the first aspect of the invention, the non-antibody anti-inflammatory factor is an inflammatory cytokine antagonist or an anti-microbial factor. In some embodiments, the inflammatory cytokine antagonist is LL-37 or thymosin β4. In some embodiments, the anti-microbial factor is LL-37 or thymosin β4.

In several embodiments of the first aspect of the invention, the stem cell recruitment factor is TGF-β3, stromal cell-derived factor (SDF)-1-α, or thymosin β4. In some embodiments, the extracellular matrix factor is collagen, laminin, or fibronectin.

In several embodiments of the first aspect of the invention, the HUCPVC synthesizes and secretes the wound healing agent.

In several embodiments of the first aspect of the invention, the HUCPVC has been genetically modified to express two or more wound-healing agents.

In several embodiments of the first aspect of the invention, the HUCPVC has been genetically modified by viral transduction, transfection, dendrimers, gene editing, or a combination thereof. In some embodiments, viral transduction includes adenoviral transduction, adeno-associated viral (AAV) transduction, or retroviral transduction. In some embodiments, the retroviral transduction is lentiviral transduction. In some embodiments, the transfection includes naked nucleic acid transfection, electroporation, gene gun transfection, lipoplex transfection, or polyplex transfection. In some embodiments, the gene editing includes clustered regularly interspaced short palindromic repeats (CRISPR)-Cas gene editing, transcription activator-like effector based nuclease (TALEN) gene editing, zinc-finger nuclease (ZFN) gene editing, or meganuclease gene editing.

In several embodiments of the first aspect of the invention, the wound healing agent is endogenous to the HUCPVC. In other embodiments, the wound healing agent is not endogenous to the HUCPVC.

In several embodiments of the first aspect of the invention, the HUCPVCs have a 3G5+, CD45−, CD44+ phenotype.

In several embodiments of the first aspect of the invention, the wound healing agent is a wild-type wound healing agent or a variant wound healing agent. In some embodiments, the variant wound healing agent is a fusion protein. In some embodiments, the fusion protein includes a fusion partner selected from a targeting moiety (e.g., a CAR peptide (CARSKNKDC, SEQ ID NO: 1)) and a detectable moiety (e.g., an epitope tag or a fluorescent protein).

In a second aspect, the invention features a composition that includes the soluble fraction of medium conditioned by the HUCPVC of the first aspect of the invention. In some embodiments, the composition includes the wound healing agent. In some embodiments, the composition includes one or more additional soluble factors produced by the genetically modified HUCPVC. In some embodiments, the one or more soluble factors are paracrine factors. In certain embodiments, the HUCPVC is grown under substantially serum-free conditions. In some embodiments, the HUCPVC is grown under substantially serum-free conditions for one or more passages.

In a third aspect, the invention features a pharmaceutical composition that includes the HUCPVC of the first aspect of the invention and a pharmaceutically acceptable carrier or excipient.

In a fourth aspect, the invention features a pharmaceutical composition that includes the composition of the second aspect of the invention and a pharmaceutically acceptable carrier or excipient. In some embodiments, the pharmaceutical composition further includes an additional therapeutic agent. In certain embodiments, the additional therapeutic agent is selected from an anti-microbial agent, an anti-inflammatory compound, a cytokine or growth factor, an analgesic, or an immunosuppressant.

In a fifth aspect, the invention features a method of treating a wound in a subject in need thereof, the method including administering a therapeutically effective amount of the pharmaceutical composition of the third aspect of the invention or of the fourth aspect of the invention to the subject.

In a sixth aspect, the invention features a method of treating a wound in a subject in need thereof, the method including administering to the subject a therapeutically effective amount of a pharmaceutical composition including (i) a genetically modified HUCPVC or (ii) a composition including the soluble fraction of medium conditioned by a genetically modified HUCPVC, wherein the HUCPCV has been genetically modified to express a wound healing agent.

In several embodiments of the sixth aspect of the invention, the wound healing agent is selected from the group consisting of an anti-fibrotic factor, an anti-inflammatory factor, a stem cell recruitment factor, an extracellular matrix factor, a cytokine or growth factor, a clotting factor, and an angiogenic factor.

In some embodiments of the sixth aspect of the invention, the anti-fibrotic factor is a TGF-β antagonist. In some embodiments, the TGF-β antagonist is an anti-TGF-β antibody or a non-antibody TGF-β antagonist. In certain embodiments, the non-antibody TGF-β antagonist is decorin.

In some embodiments of the sixth aspect of the invention, the anti-inflammatory factor is an inflammatory cytokine antagonist or an anti-microbial factor. In some embodiments, the inflammatory cytokine antagonist is IL-10, LL-37, or thymosin β4. In some embodiments, the inflammatory cytokine antagonist is an antibody. In certain embodiments, the antibody is an anti-TNF-α antibody, an anti-IL-6 antibody, or an anti-IL-10 antibody. In some embodiments, the anti-microbial factor is LL-37 or thymosin β4.

In some embodiments of the sixth aspect of the invention, the stem cell recruitment factor is TGF-β3, stromal cell-derived factor (SDF)-1-α, or thymosin β4.

In some embodiments of the sixth aspect of the invention, the extracellular matrix factor is collagen, laminin, or fibronectin.

In some embodiments of the sixth aspect of the invention, the cytokine or growth factor is selected from the group consisting of interleukins (ILs), epidermal growth factor (EGF), fibroblast growth factors (FGFs), platelet-derived growth factors (PDGFs), keratinocyte growth factor (KGF), bone morphogenetic proteins (BMPs), and colony stimulating factors (CSFs). In certain embodiments, the interleukin is IL-2 or IL-10. In some embodiments, the FGF is FGF-1, FGF-2, FGF-7, or FGF-10. In certain embodiments, the BMP is selected from the group consisting of BMP-2, BMP-4, BMP-6, and BMP-7. In certain embodiments, the CSF is GM-CSF.

In some embodiments of the sixth aspect of the invention, the clotting factor is selected from factor I, factor II, CD142, factor V, factor VII, factor VIII, factor IX, factor X, factor XI, factor XII, factor XIII, von Willebrand factor, prekallikrein, high-molecular weight kininogen (HMWK), fibronectin, antithrombin III, heparin cofactor II, protein C, protein S, protein Z, plasminogen, tissue plasminogen activator (tPA), and urokinase.

In some embodiments of the sixth aspect of the invention, the angiogenic factor is a vascular endothelial growth factor (VEGF) or an angiopoetin. In certain embodiments, the VEGF is selected from the group consisting of VEGF-A, VEGF-B, VEGF-C, VEGF-D, VEGF-E, and placental growth factor (PIGF). In certain embodiments, the angiopoietin is ANGPT1 or ANGPT2.

In several embodiments of the sixth aspect of the invention, the HUCPVC synthesizes and secretes the wound healing agent.

In several embodiments of the sixth aspect of the invention, the HUCPVC has been genetically modified to express two or more wound-healing agents.

In several embodiments of the sixth aspect of the invention HUCPVC has been genetically modified by viral transduction, transfection, dendrimers, gene editing, or a combination thereof. In some embodiments, the viral transduction includes adenoviral transduction, AAV transduction, or retroviral transduction. In certain embodiments, the retroviral transduction is lentiviral transduction. In some embodiments, the transfection includes naked nucleic acid transfection, electroporation, gene gun transfection, lipoplex transfection, or polyplex transfection. In some embodiments, the gene editing includes CRISPR-Cas gene editing, transcription activator-like effector based nuclease (TALEN) gene editing, zinc-finger nuclease (ZFN) gene editing, or meganuclease gene editing.

In several embodiments of the sixth aspect of the invention, the wound healing agent is endogenous to the HUCPVC. In other embodiments, the wound healing agent is not endogenous to the HUCPVC.

In several embodiments of the sixth aspect of the invention, the HUCPVCs have a 3G5+, CD45−, CD44+ phenotype.

In several embodiments of the sixth aspect of the invention, the wound healing agent is a wild-type wound healing agent or a variant wound healing agent. In some embodiments, the variant wound healing agent is a fusion protein. In some embodiments, the fusion protein includes a fusion partner selected from a targeting moiety and a detectable moiety. In certain embodiments, the targeting moiety includes a CAR peptide (CARSKNKDC, SEQ ID NO: 1). In some embodiments, the detectable moiety is an epitope tag or a fluorescent protein.

In several embodiments of the fifth aspect of the invention and of the sixth aspect of the invention, the subject is a vertebrate. In some embodiments, the vertebrate is a mammal. In certain embodiments, the mammal is a human.

In several embodiments of the fifth aspect of the invention and of the sixth aspect of the invention, the genetically modified HUCPVC is allogeneic or xenogeneic to the subject.

In several embodiments of the fifth aspect of the invention and of the sixth aspect of the invention, the method includes administering a single dose of the pharmaceutical composition. In other embodiments, the method includes administering multiple doses of the pharmaceutical composition.

In several embodiments of the fifth aspect of the invention and of the sixth aspect of the invention, the genetically modified HUCPVC persists in the subject for greater than one week. In some embodiments, the genetically modified HUCPVC persists in the subject for greater than one month. In certain embodiments, the genetically modified HUCPVC persists in the subject for greater than two months.

In several embodiments of the fifth aspect of the invention and of the sixth aspect of the invention, the pharmaceutical composition is administered to the subject intravenously, intramuscularly, subcutaneously, orally, by inhalation, parenterally, intraperitoneally, intraarterially, transdermally, sublingually, nasally, transbuccally, liposomally, adiposally, opthalmically, intraocularly, intrathecally, topically, or locally.

In several embodiments of the fifth aspect of the invention and of the sixth aspect of the invention, the HUCPVC evades immune recognition in the subject.

In several embodiments of the fifth aspect of the invention and of the sixth aspect of the invention, the subject is administered between 10¹ and 10¹³ HUCPVCs per dose. In some embodiments, the subject is administered between 10³ and 10⁸ HUCPVCs per dose.

In several embodiments of the fifth aspect of the invention and of the sixth aspect of the invention, the method further includes administering at least one mesenchymal stem cell (MSC), wherein the MSC is not a HUCPVC. In some embodiments, the MSC has been genetically modified to express a wound healing agent. In some embodiments, the wound healing agent is selected from an anti-fibrotic factor, an anti-inflammatory factor, a stem cell recruitment factor, an extracellular matrix factor, a cytokine or growth factor, a clotting factor, and an angiogenic factor. In certain embodiments, the MSC is isolated from bone marrow, umbilical cord blood, embryonic yolk sac, placenta, skin, or blood.

In several embodiments of the fifth aspect of the invention and of the sixth aspect of the invention, the method further includes administering one or more additional therapeutic agents to the subject. In some embodiments, the one or more additional therapeutic agents enhances or prolongs the therapeutic benefit of the HUCPVC treatment. In some embodiments, the one or more additional therapeutic agents is selected from the group consisting of an anti-microbial agent, an anti-inflammatory compound, a cytokine or growth factor, an analgesic, or an immunosuppressant.

In several embodiments of the fifth aspect of the invention and of the sixth aspect of the invention, the wound is an open wound, a closed wound, a chronic wound, or a burn. In some embodiments, the open wound is selected from the group consisting of an incision, a laceration, an abrasion, an avulsion, a puncture wound, a penetration wound, and a gunshot wound. In some embodiments, the closed wound is a hematoma or a crush injury. In some embodiments, the chronic wound is a venous ulcer, a diabetic ulcer, or a pressure ulcer. In certain embodiments, the diabetic ulcer is a diabetic foot ulcer.

In a seventh aspect, the invention features a method for producing a genetically modified HUCPVC, the method including introducing a nucleic acid encoding a wound healing agent into a HUCPVC, thereby producing a genetically modified HUCPVC expressing a wound healing agent.

In several embodiments of the seventh aspect of the invention, the wound healing agent is selected from the group consisting of an anti-fibrotic factor, an anti-inflammatory factor, a stem cell recruitment factor, an extracellular matrix factor, a cytokine or growth factor, a clotting factor, and an angiogenic factor.

In some embodiments of the seventh aspect of the invention, the anti-fibrotic factor is a TGF-β antagonist. In some embodiments, the TGF-β antagonist is an anti-TGF-β antibody or a non-antibody TGF-β antagonist. In certain embodiments, the non-antibody TGF-β antagonist is decorin.

In some embodiments of the seventh aspect of the invention, the anti-inflammatory factor is an inflammatory cytokine antagonist or an anti-microbial factor. In some embodiments, the inflammatory cytokine antagonist is IL-10, LL-37, or thymosin β4. In some embodiments, the inflammatory cytokine antagonist is an antibody. In certain embodiments, the antibody is an anti-TNF-α antibody, an anti-IL-6 antibody, or an anti-IL-10 antibody. In some embodiments, the anti-microbial factor is LL-37 or thymosin β4.

In some embodiments of the seventh aspect of the invention, the stem cell recruitment factor is TGF-β3, stromal cell-derived factor (SDF)-1-α, or thymosin β4.

In some embodiments of the seventh aspect of the invention, the extracellular matrix factor is collagen, laminin, or fibronectin.

In some embodiments of the seventh aspect of the invention, the cytokine or growth factor is selected from the group consisting of interleukins (ILs), epidermal growth factor (EGF), fibroblast growth factors (FGFs), platelet-derived growth factors (PDGFs), keratinocyte growth factor (KGF), bone morphogenetic proteins (BMPs), and colony stimulating factors (CSFs). In certain embodiments, the interleukin is IL-2 or IL-10. In some embodiments, the FGF is FGF-1, FGF-2, FGF-7, or FGF-10. In certain embodiments, the BMP is selected from the group consisting of BMP-2, BMP-4, BMP-6, and BMP-7. In certain embodiments, the CSF is GM-CSF.

In some embodiments of the seventh aspect of the invention, the clotting factor is selected from factor I, factor II, CD142, factor V, factor VII, factor VIII, factor IX, factor X, factor XI, factor XII, factor XIII, von Willebrand factor, prekallikrein, high-molecular weight kininogen (HMWK), fibronectin, antithrombin III, heparin cofactor II, protein C, protein S, protein Z, plasminogen, tissue plasminogen activator (tPA), and urokinase.

In some embodiments of the seventh aspect of the invention, the angiogenic factor is a vascular endothelial growth factor (VEGF) or an angiopoietin. In certain embodiments, the VEGF is selected from the group consisting of VEGF-A, VEGF-B, VEGF-C, VEGF-D, VEGF-E, and placental growth factor (PIGF). In certain embodiments, the angiopoietin is ANGPT1 or ANGPT2.

In several embodiments of the seventh aspect of the invention, the HUCPVC synthesizes and secretes the wound healing agent.

In several embodiments of the seventh aspect of the invention, the HUCPVC is genetically modified to express two or more wound-healing agents.

In several embodiments of the seventh aspect of the invention, the nucleic acid is introduced into the HUCPVC by viral transduction, transfection, dendrimers, gene editing, or a combination thereof. In some embodiments, the viral transduction includes adenoviral transduction, AAV transduction, or retroviral transduction. In certain embodiments, the retroviral transduction is lentiviral transduction. In some embodiments, the transfection includes naked nucleic acid transfection, electroporation, gene gun transfection, lipoplex transfection, or polyplex transfection. In some embodiments, the gene editing includes CRISPR-Cas gene editing, transcription activator-like effector based nuclease (TALEN) gene editing, zinc-finger nuclease (ZFN) gene editing, or meganuclease gene editing.

In several embodiments of the seventh aspect of the invention, the wound healing agent is endogenous to the HUCPVC. In other embodiments, the wound healing agent is not endogenous to the HUCPVC.

In several embodiments of the seventh aspect of the invention, the HUCPVCs have a 3G5+, CD45−, CD44+ phenotype.

In several embodiments of the seventh aspect of the invention, the wound healing agent is a wild-type wound healing agent or a variant wound healing agent. In some embodiments, the variant wound healing agent is a fusion protein. In some embodiments, the fusion protein includes a fusion partner selected from a targeting moiety and a detectable moiety. In certain embodiments, the targeting moiety includes a CAR peptide (CARSKNKDC, SEQ ID NO: 1). In some embodiments, the detectable moiety is an epitope tag or a fluorescent protein.

In an eighth aspect, the invention features a method of treating a wound, the method including administering a therapeutically effective amount of a pharmaceutical composition including the soluble fraction of medium conditioned by a HUCPVC, wherein the HUCPVC has been grown for one or more passages under substantially serum-free conditions.

In some embodiments of the eighth aspect of the invention, the pharmaceutical composition includes one or more additional soluble factors produced by the HUCPVC. In certain embodiments, the one or more soluble factors are paracrine factors.

In several embodiments of the eighth aspect of the invention, the subject is a vertebrate. In some embodiments, the vertebrate is a mammal. In certain embodiments, the mammal is a human.

In some embodiments of the eighth aspect of the invention, the method includes administering a single dose of the pharmaceutical composition. In other embodiments, the method includes administering multiple doses of the pharmaceutical composition.

In several embodiments of the eighth aspect of the invention, the pharmaceutical composition is administered to the subject intravenously, intramuscularly, subcutaneously, orally, by inhalation, parenterally, intraperitoneally, intraarterially, transdermally, sublingually, nasally, transbuccally, liposomally, adiposally, opthalmically, intraocularly, intrathecally, topically, or locally.

In several embodiments of the eighth aspect of the invention, the method further includes administering at least one MSC or HUCPVC. In some embodiments, the MSC or HUCPVC has been genetically modified to express a wound healing agent. In some embodiments, the wound healing agent is selected from an anti-fibrotic factor, an anti-inflammatory factor, a stem cell recruitment factor, an extracellular matrix factor, a cytokine or growth factor, a clotting factor, and an angiogenic factor. In certain embodiments, the MSC is isolated from bone marrow, umbilical cord blood, embryonic yolk sac, placenta, skin, or blood. In some embodiments, the wound healing agent is decorin.

In several embodiments of the eighth aspect of the invention, the method further includes administering one or more additional therapeutic agents to the subject. In some embodiments the one or more additional therapeutic agents enhances or prolongs the therapeutic benefit of the HUCPVC treatment. In certain embodiments, the one or more additional therapeutic agents is selected from the group consisting of an anti-microbial agent, an anti-inflammatory compound, a cytokine or growth factor, an analgesic, or an immunosuppressant.

In several embodiments of the eighth aspect of the invention, the wound is an open wound, a closed wound, a chronic wound, or a burn. In some embodiments, the open wound is selected from the group consisting of an incision, a laceration, an abrasion, an avulsion, a puncture wound, a penetration wound, and a gunshot wound. In some embodiments, the closed wound is a hematoma or a crush injury. In some embodiments, the chronic wound is a venous ulcer, a diabetic ulcer, or a pressure ulcer. In certain embodiments the diabetic ulcer is a diabetic foot ulcer.

### Definitions

It is to be understood that aspects and embodiments of the invention described herein include “comprising,” “consisting,” and “consisting essentially of” aspects and embodiments. As used herein, the singular form “a,” “an,” and “the” includes plural references unless indicated otherwise.

The term “about” is used herein to mean a value that is ±10% of the recited value.

The term “administering,” as used herein, refers to a method of giving a dosage of a composition described herein (e.g., a genetically modified HUCPVC, medium conditioned by a HUCPVC (e.g., a genetically modified HUCPVC), compositions that include the soluble fraction of such medium, and pharmaceutical compositions thereof) to a subject. The compositions utilized in the methods described herein can be administered, for example, intravenously, intramuscularly, subcutaneously, orally, by inhalation, parenterally, intraperitoneally, intraarterially, transdermally, sublingually, nasally, transbuccally, liposomally, adiposally, opthalmically, intraocularly, intrathecally, topically, or locally. Administration can be systemic or local. The preferred method of administration can vary depending on, for example, the components of the composition being administered and the severity of the condition (e.g., the wound) being treated.

The term “antibody,” as used herein, includes whole antibodies or immunoglobulins and any antigen-binding fragment or single chains thereof. Antibodies, as used herein, can be mammalian (e.g., human or mouse), humanized, chimeric, recombinant, synthetically produced, or naturally isolated. Antibodies of the present invention include all known forms of antibodies and other protein scaffolds with antibody-like properties. For example, the antibody can be a human antibody, a humanized antibody, a bispecific antibody, a chimeric antibody, or a protein scaffold with antibody-like properties, such as fibronectin or ankyrin repeats. The antibody also can be a Fab, F(ab′)₂, scFv, SMIP, diabody, nanobody, aptamers, or a domain antibody. The antibody can have any of the following isotypes: IgG (e.g., IgG1, IgG2, IgG3, and IgG4), IgM, IgA (e.g., IgA1, IgA2, and IgAsec), IgD, or IgE.

The term “antibody fragment,” as used herein, refers to one or more fragments of an antibody that retain the ability to specifically bind to an antigen. The antigen-binding function of an antibody can be performed by fragments of a full-length antibody. Examples of binding fragments encompassed within the term “antigen-binding portion” of an antibody include but are not limited to: (i) a Fab fragment, a monovalent fragment consisting of the V_{L}, V_{H}, C_{L}, and C_{H}1 domains; (ii) a F(ab′)₂ fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546 (1989)), which consists of a V_{H} domain; (vii) a dAb which consists of a V_{H} or a V_{L} domain; (viii) an isolated complementarity determining region (CDR); and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al., Science 242:423-426 (1988) and Huston et al., Proc. Natl. Acad. Sci. USA 85:5879-5883 (1988)).

The terms “effective amount,” “amount effective to,” and “therapeutically effective amount” mean an amount of genetically modified HUCPVCs, medium conditioned by HUCPVCs (e.g., genetically modified HUCPVCs), and/or a composition that includes the soluble fraction of such medium that is sufficient to produce a desired result, for example, treating a wound.

As used herein, the terms “express” and “expression” refer to the process by which information (e.g., genetic and/or epigenetic information) is converted into the structures present in a cell (e.g., a HUCPCV) or secreted therefrom. Accordingly, as used herein, “expression” may refer to transcription, translation, or polynucleotide and/or polypeptide modifications (e.g., posttranslational modification of a polypeptide).

By “genetically modified HUCPVC” is meant a human umbilical cord perivascular cell that recombinantly expresses at least one wound healing agent that, when administered to a subject (e.g., a human), can treat a wound or a symptom associated with a wound. The wound healing agent is recombinantly produced by the HUCPVC following transfer (e.g., transfection, transduction, or gene editing) of the genetic sequence(s) encoding the wound healing agent to the HUCPVC.

The term “human antibody,” as used herein, is intended to include antibodies, or fragments thereof, having variable regions in which both the framework and CDR regions are derived from human germline immunoglobulin sequences as described, for example, by Kabat et al., (Sequences of Proteins of Immunological Interest, Fifth Edition, U.S. Department of Health and Human Services, NIH Publication No. 91-3242 (1991)). Furthermore, if the antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences (i.e., a humanized antibody or antibody fragment).

The term “humanized antibody” refers to any antibody or antibody fragment that includes at least one immunoglobulin domain having a variable region that includes a variable framework region substantially derived from a human immunoglobulin or antibody and complementarity determining regions (e.g., at least one CDR) substantially derived from a non-human immunoglobulin or antibody.

The term “parenteral” as used herein refers to subcutaneous, intracutaneous, intravenous, intraperitoneal, intramuscular, intraarticular, intraarterial, intrasynovial, intrasternal, intrathecal, intralesional, or intracranial administration (e.g., injection), as well as any suitable infusion technique.

By “pharmaceutically acceptable carrier” is meant a carrier which is physiologically acceptable to the treated subject (e.g., a human) while retaining the therapeutic properties of the genetically modified HUCPVCs with which it is administered. One exemplary pharmaceutically acceptable carrier is physiological saline. Other physiologically acceptable carriers and their formulations are known to one skilled in the art and described, for example, in Remington's Pharmaceutical Sciences, (18^{th} edition), ed. A. Gennaro, 1990, Mack Publishing Company, Easton, Pa. incorporated herein by reference.

By “pharmaceutical composition” is meant any composition that contains a therapeutically or biologically active agent (e.g., a genetically modified HUCPVC, a medium conditioned by a HUCPVC (e.g., a genetically modified HUCPVC), or a composition that includes the soluble fraction of such a medium) that is suitable for administration to a subject (e.g., a human).

By “treating” is meant a reduction (e.g., by at least about 10%, about 15%, about 20%, about 25%, about 30%, about 35%, about 40%, about 45%, about 50%, about 60%, about 70%, about 80%, about 90%, about 95%, about 99%, or even 100%) in the progression or severity of a disease or disorder (e.g., a wound), or in the progression, severity, or frequency of one or more symptoms of the disease or disorder (e.g., a wound) in a subject (e.g., a human).

The term “wound healing agent,” as used herein, refers to a biological agent that is involved in or that affects (e.g., promotes) wound healing, including, without limitation, nucleic acids (e.g., DNA and RNA (e.g., mRNAs and small interfering RNAs (siRNAs)), polypeptides (including glycoproteins (e.g., proteoglycans)), and hormones. In some embodiments, the wound healing agent may be an anti-fibrotic factor, an anti-inflammatory factor (e.g., a non-antibody inflammatory factor), a stem cell recruitment factor, an extracellular matrix factor, a cytokine or growth factor, a clotting factor, or an angiogenic factor. The wound healing agent may be a wild-type wound healing agent or an engineered wound healing agent (e.g., a variant wound healing agent having one or more mutations (e.g., point mutations, insertions, or deletions) or a wound healing fusion protein). A wound healing fusion protein may include, for example, a targeting moiety (e.g., a CAR peptide, CARSKNKDC, SEQ ID NO: 1) or a detectable moiety (e.g., an epitope tag (e.g., myc, HA, and the like) or a fluorescent protein (e.g., green fluorescent protein (GFP), red fluorescent protein (RFP), yellow fluorescent protein (YFP), and variants thereof).

The term “fibrosis” refers to the formation of fibrous tissue, usually as a reparative or a reactive process, such as during wound healing. As used herein, “fibrosis” includes those disorders or disease states that are caused by or accompanied by the abnormal deposition of scar tissue, or by excessive accumulation of collagenous connective tissue. Fibrosis may occur in any organ including, for example, skin, kidney, lung, liver, the central nervous system, bone, bone marrow, the cardiovascular system, an endocrine organ, or the gastrointestinal system.

The term “anti-fibrotic factor” refers to any biological agent that inhibits or reduces fibrosis, which in the context of wound healing can lead to scarring. Anti-fibrotic factors may have different mechanisms of action, including, for example, reducing the formation of extracellular matrix proteins (e.g., collagen), enhancing the metabolism or removal of extracellular matrix proteins (e.g., collagen) in the affected area of the body, or promoting proper organization of extracellular matrix proteins (e.g., collagen). In some embodiments, an anti-fibrotic factor may be a TGF-β antagonist (e.g., a TGF-β1 antagonist or a TGF-β2 antagonist). In some embodiments, a TGF-β antagonist may be decorin. In some embodiments, the decorin may be a part of a fusion protein, for example, CAR-decorin, which includes a wound homing peptide, CAR (CARSKNKDC, SEQ ID NO: 1), see, e.g., Jirvinen and Ruoslahti, “Target-seeking antifibrotic compound enhances wound healing and suppresses scar formation in mice,” Proc. Natl. Acad. Sci. USA 107(50):21671-21676 (2010) and U.S. Pat. No. 9,180,161. In other embodiments, a TGF-β antagonist may be, for example, an anti-TGF-β antibody (e.g., fresolimumab, lerdelimumab, and metelimumab) or an anti-TGF-β oligonucleotide (e.g., trabedersen (an antisense oligonucleotide targeting TGF-β2) or an siRNA targeting TGF-β). The anti-TGF-β antibody may be a monoclonal antibody, a humanized antibody, or a human antibody. The anti-TGF-β antibody may be an antibody fragment.

The term “anti-fibrotic factor” also encompasses any suitable anti-fibrotic factor known in the art, including, for example, cathepsin D, cathepsin E, cathepsin S, cathepsin K, cathepsin L, cathepsin B, cathespin C, cathepsin H, cathespin F, cathepsin G, cathepsin O, cathepsin R, cathepsin V (cathepsin 12), cathepsin W, cathepsin Z (cathepsin X), calpin 1, calpin 2, chondroitinase ABC, chondroitinase AC, pancreatic elastase, elastase-2a, elastase-2b, neutrophil elastase, proteinase-3, endogenous vascular elastase, mast cell chymase, mast cell tryptase, plasmin, thrombin, granzyme B, hyaluronidase, chymopapain, chymotrypsin, legumain, collagenase, matrix metalloproteinases (e.g., MMP-1 (collagenase-1), MMP-9, MMP-7 (matrilysin), MMP-8 (collagenase-2), MMP-13 (collagenase-3), MMP-18 (collagenase-4), MMP-2 (gelatinase a), MMP-9 (gelatinase b), MMP-3 (stromelysin-1), MMP-10 (stromelysin-2), MMP-11 (stromelysin-3), MMP-7 (matrilysin), MMP-26 (matrilysin), MMP-12 (metalloelastase), MMP-14 (MT1-MMP), MMP-15 (MT2-MMP), MMP-16 (MT3-MMP), MMP-17 (MT4-MMP), MMP-24 (MT5-MMP), MMP-25 (MT6-MMP), MMP-19, MMP-20 (enamelysin), MMP-x, MMP-23, MMP-27, and MMP-28 (epilysin)), ADAMTS-1, ADAMTS-2, ADAMTS-3, ADAMTS-4 (aggrecanase-1), ADAMTS-5 (aggrecanase-2), ADAMTS-14, papain, subtilisin, subtilisin A, heparanase, TGF-β receptor antagonists (e.g., TGF-β receptor antibodies), angiotensin inhibitors, angiotensin-converting enzyme inhibitors, angiotensin-II receptor antagonists, cannabinoid receptor, or a combination thereof. In some embodiments, the anti-fibrotic factor includes, but are not limited to, interleukins, interferons (e.g., interferon gamma), cytokines, chemokines, chemotactic molecules, relaxin, hormones (e.g., progesterone, estrogen, testosterone, growth hormone, thyroid hormone, parathyroid hormone, and the like) or a combination thereof.

In some embodiments, the anti-fibrotic factor is a “non-antibody anti-fibrotic factor.” As used herein, this term specifically excludes antibodies (e.g., monoclonal antibodies, human antibodies, and humanized antibodies). In some embodiments, this definition specifically excludes anti-TGF-β antibodies, e.g., fresolimumab, lerdelimumab, and metelimumab.

The term “anti-inflammatory factor,” as used herein, refers to any biological agent that inhibits or reduces inflammation. An anti-inflammatory factor may include, for example, inflammatory cytokine antagonists (e.g., IL-6 antagonists and/or IL-10 antagonists) and anti-microbial factors. An “inflammatory cytokine antagonist” refers to any agent which decreases, blocks, inhibits, abrogates, or interferes with the pro-inflammatory cascade of cytokine proteins leading to an inflammatory response. Exemplary inflammatory cytokine antagonists include IL-10 (which in some embodiments functions as an IL-6 antagonist and/or an IL-10 antagonist), LL-37, and thymosin β4. In some embodiments, the anti-microbial factor is LL-37 or thymosin β4. In some embodiments, an anti-inflammatory agent (e.g., an inflammatory cytokine antagonist) may be an antibody (e.g., an anti-TNFα antibody (e.g., infliximab, adalimumab, certolizumab pegol, and golimumab), an anti-IL-6 antibody, or an anti-IL-10 antibody). The antibody may be a monoclonal antibody, a humanized antibody, or a human antibody. The antibody may be an antibody fragment. In other instances, an anti-inflammatory factor (e.g., an inflammatory cytokine antagonist) may be a soluble receptor fusion protein, e.g., etanercept.

In some embodiments, the anti-inflammatory factor is a “non-antibody anti-inflammatory factor.” As used herein, this term specifically excludes antibodies (e.g., monoclonal antibodies, human antibodies, and humanized antibodies). In some embodiments, the term “non-inflammatory anti-inflammatory factor” specifically excludes anti-tumor necrosis factor (TNF) antibodies (e.g., infliximab, adalimumab, certolizumab pegol), alemtuzumab, afelimomab, aselizumab, atlizumab, atorolimumab, basiliximab, belimumab, bertilimumab, cedelizumab, clenoliximab, daclizumab, dorlimomab aritox, dorlixizumab, eculizumab, efalizumab, elsilimomab, erlizumab, faralimomab, fontolizumab, galiximab, gantenerumab, gavilimomab, golimumab, gomiliximab, ibalizumab, inolimomab, ipilimumab, keliximab, lebrilizumab, lerdelimumab, lumiliximab, maslimomab, mepolizumab, metelimumab, morolimumab, muromonab-CD3, natalizumab, nerelimomab, ocrelizumab, odulimomab, omalizumab, otelixizumab, pascolizumab, pexelizumab, rituxumab, reslizumab, rovelizumab, ruplizumab, siplizumab, talizumab, telimomab aritox, teneliximab, teplizumab, tocilizumab, toralizumab, vapaliximab, vepalimomab, visilizumab, zanolimumab, ziralimumab, and zolimomab aritox.

A “stem cell recruitment factor” is a biological agent that can promote the migration, maintenance, and/or proliferation of endogenous stem cells to a particular location in the body of a subject, for example, a wound. Non-limiting examples of stem cell recruitment factors include TGF-β3, stromal cell-derived factor (SDF)-1-α, and thymosin β4.

As used herein, an “extracellular matrix factor” refers to a component of the extracellular matrix or a regulator thereof that is involved in wound healing. Non-limiting examples of extracellular matrix factors include collagen (e.g., collagen-I, collagen-III, and collagen-VI), decorin, fibronectin, vitronectin, laminin, cartilage oligomeric matrix protein (COMP), tenascin-C, tenascin-X, elastin, keratin (e.g., K6 and K16), tissue inhibitor of metalloproteinase-1 (TIMP-1), albumin, osteonectin, thrombospondin (e.g., thrombospondin-1 or thrombospondin-2), proteoglycans (e.g., versican, syndecan, glypicans, perlecan, lumican, and heparin sulfate), glycosaminoglycans (e.g., hyaluranon/hyaluronic acid), and integrins.

A “clotting factor” refers to a biological agent that is involved in clotting (also known as coagulation). As used herein, this term encompasses agents involved in platelet activation as well as the coagulation cascade (including both the intrinsic and extrinsic pathways) that leads to fibrin formation. Clotting factors include, but are not limited to, factor I (fibrinogen/fibrin), factor II (prothrombin), CD142 (also known as tissue factor, tissue thromboplastin, or factor III), factor V, factor VII, factor VIII, factor IX, factor X, factor XI, factor XII, factor XIII, von Willebrand factor, prekallikrein, high-molecular weight kininogen (HMWK), fibronectin, antithrombin III, heparin cofactor II, protein C, protein S, protein Z, plasminogen, tissue plasminogen activator (tPA), and urokinase.

As used herein, an “angiogenic factor” is a biological agent that is involved in angiogenesis, the formation of new blood vessels. Angiogenic factors include, but are not limited to, vascular endothelial growth factors (VEGFs), including, e.g., VEGF-A, VEGF-B, VEGF-C, VEGF-D, VEGF-E, and placental growth factor (PIGF); angiopoietins and angiopoietin-like proteins (e.g., ANGPT1, ANGPT2, ANGPT4, ANGPTL1, ANGPTL2, ANGPTL3, ANGPTL4, ANGPTL5, ANGPTL6, and ANGTPL7); fibroblast growth factors (FGFs), including FGF-1 and FGF-2; epidermal growth factor (EGF); transforming growth factors (TGFs), including TGF-α and TGF-β, tumor necrosis factors (TNFs), including TNF-α; colony stimulating factors (CSFs), including granulocyte macrophage colony-stimulating factor (GM-CSF) and macrophage colony stimulating factor (M-CSF, also known as CSF1); and nitric oxide synthase (NOS).

As used herein, “substantially serum-free conditions” means that the culture medium of HUCPVCs contains, for example, less than about 10% serum (e.g., less than about 9% serum, less than about 8% serum, less than 5% serum, less than about 2% serum, less than about 1% serum, less than about 0.5% serum, and less than about 0.1% serum). The percentage may be a volume/volume (v/v) percentage. The term may indicate that the culture medium contains only trace amounts of serum. The term also encompasses the absence of serum (e.g., exogenously added serum).

It is to be understood that the foregoing lists of wound healing agents are not all-inclusive, and that in some instances, a protein may belong to more than one class of wound healing agents. For example, in some instances, a specific growth factor or cytokine may be classified as either an anti-fibrotic agent or an anti-inflammatory agent. In another example, decorin may be classified as an anti-fibrotic factor or an extracellular matrix factor.

Other features and advantages of the invention will be apparent from the following detailed description, and from the claims.

### BRIEF DESCRIPTION OF THE DRAWINGS

FIG. 1 is a graph showing the results of microarray analysis to determine the expression level of decorin (dcn) in HUCPVCs. The graph plots signal intensity (i.e., expression level) as a function of the passage of the HUCPVCs.

FIG. 2 is a graph showing the results of an enzyme linked immunosorbent assay (ELISA) demonstrating that HUCPVCs secrete decorin (Dcn) into the culture medium, and that HUCPVCs can be genetically modified to express and secrete Dcn at levels higher than wild-type, unmodified HUCPVCs. This graph also shows that HUCPVCs can be genetically modified to efficiently express and secrete CAR-Dcn. The graph plots Dcn levels (ng/ml/million cells) as a function of time (days post-engineering). “eDCN-HUCPVC” indicates HUCPVCs engineered with the human Dcn gene. “eDCN-CAR-HUCPVC” indicates HUCPVCs engineered with a transgene encoding Dcn fused to the CAR peptide. “MOI” is an abbreviation for multiplicity of infection (the ratio of infective recombinant adenovirus to cell number at transfection, where a higher MOI typically produces higher transgene copy number per cell).

FIG. 3 is a graph showing that HUCPVCs secrete more Dcn as a result of higher transgene copy number. CM was collected from 72 hour cultures and analyzed for decorin protein by ELISA. Dcn was detected in media from native HUCPVCs, and at step-wise higher concentrations correlating to predicted Dcn transgene copy number. The graph plots Dcn levels (ng/ml/million cells) as a function of time (days post-engineering). Data represents averages from a minimum of two HUCPVC donor lots.

FIG. 4 is an image of a Western blot showing that HUCPVCs secrete homogenous Dcn and CAR-Dcn products into CM. Proteins from conditioned media samples analyzed by ELISA (see FIGS. 2 and 3) were diluted 1:10, separated by denaturing sodium dodecyl sulfate (SDS) gel electrophoresis, transferred to a PVDF membrane, and probed with an antibody against human Dcn. Dcn was detected as a sharp band in all experimental lanes, indicating a homogeneous protein population. “PSL” is an abbreviation for pre-stained ladder. “MML” is an abbreviation for MagicMark™ Western blot ladder. “DCN” indicates purified Dcn protein standard. “N” indicates native HUCPVCs. “D20” indicates Dcn-engineered HUCPVCs at MOI 20. “D100” indicates Dcn-engineered HUCPVCs at MOI 100. “C20” indicates CAR-Dcn-engineered HUCPVCs at M0120. “C100” indicates CAR-Dcn-engineered HUCPVCs at MOI 100. “E” indicates empty. “M” indicates medium alone.

FIGS. 5A-5F are a series of images showing that conditioned media from native and Dcn-engineered HUCPVCs affects migration of human dermal fibroblasts to close an in vitro wound. Monolayers of human dermal fibroblasts were scratched to create a linear wound, then incubated with either media alone (FIG. 5A), media supplemented with Dcn protein, or conditioned media from native and Dcn-engineered HUCPVCs. Treatment with Dcn alone (FIGS. 5B and 5C) promoted individual fibroblasts to migrate from the wound margin into the gap. CM from native HUCPVCs (FIG. 5D) stimulated the wound margins to close the gap as a sheet. CM from Dcn-engineered HUCPVCs (FIGS. 5E and 5F) produced an intermediate response, with some migration of the fibroblast sheet, but evidence of more individual fibroblasts than in FIG. 5C.

FIGS. 6A-6H are a series of images showing that conditioned media from native and Dcn-engineered HUCPVCs affects migration of human dermal fibroblasts to close an in vitro wound in a similar manner to co-culture with native and Dcn-engineered HUCPVCs. Monolayers of human dermal fibroblasts were scratched to create a linear wound, then co-cultured with media alone or native and Dcn-engineered HUCPVCs (FIGS. 6A-6D). Duplicate experiments were performed using CM from the same cell samples (FIGS. 6E-6H). Engineering with Dcn altered the wound closure phenotype produced by native HUCPVCs, and this effect was more pronounced in CM-incubated cultures than in live cell co-cultures.

### DETAILED DESCRIPTION OF THE INVENTION

The invention features human umbilical cord perivascular cells (HUCPVCs) that are genetically modified (e.g., to express a wound healing agent); medium conditioned by HUCPVCs (including genetically modified HUCPVCs); compositions that include the soluble fraction of medium conditioned by HUCPVCs (including genetically modified HUCPVCs); pharmaceutical compositions that include genetically modified HUCPVCs, medium conditioned by HUCPVCs (e.g., genetically modified HUCPVCs), and/or compositions that include the soluble fraction of such medium; and methods of use thereof for treatment of wounds.

A HUCPVC can be genetically modified to express a wound healing agent, for example, an anti-fibrotic factor, an anti-inflammatory factor (e.g., a non-antibody inflammatory factor), a stem cell recruitment factor, an extracellular matrix factor, a cytokine or growth factor, a clotting factor, or an angiogenic factor. Genetically modified HUCPVCs can be administered to subjects (e.g., humans) at risk of, or suffering from, a wound to provide prophylactic or therapeutic benefit to the treated subject. A HUCPVC may be genetically modified to express one wound healing agent or more than one (e.g., two, three, four, five, six, seven, eight, nine, ten, or more) wound healing agents.

The medium conditioned by HUCPVCs (e.g., genetically modified HUCPVCs) is also a useful composition for prophylaxis or treatment of a subject at risk of, or suffering from, a wound. HUCPVCs are considered to secrete a number of soluble factors into the medium that have beneficial properties for wound healing. Additionally, in the case of HUCPVCs that are genetically modified to express one or more wound healing agents, the conditioned medium may contain the one or more wound healing agents as well as additional soluble factors secreted by HUCPVCs. In some embodiments, the HUCPVCs are cultured in substantially serum-free conditions to obtain conditioned medium. In some embodiments, a composition comprising the soluble fraction (or a subfraction thereof) may be used for prophylaxis or treatment of a subject at risk of, or suffering from, a wound.

Genetically modified HUCPVCs (e.g., genetically modified to express a wound healing agent), medium conditioned by HUCPVCs (e.g., genetically modified HUCPVCs), and/or compositions that include the soluble fraction of medium conditioned by HUCPVCs (e.g., genetically modified HUCPVCs) can be co-administered with one or more diagnostic or therapeutic agents, for example, to enhance or prolong the prophylactic or therapeutic qualities of the treatment. HUCPVCs, medium conditioned by HUCPVCs (e.g., genetically modified HUCPVCs), and/or compositions that include the soluble fraction of medium conditioned by HUCPVCs (e.g., genetically modified HUCPVCs) can also be combined with one or more pharmaceutically acceptable carriers or excipients and can be formulated to be administered by any suitable route, for example, intravenously, intramuscularly, orally, by inhalation, parenterally, intraperitoneally, intraarterially, transdermally, sublingually, nasally, transbuccally, liposomally, adiposally, opthalmically, intraocularly, subcutaneously, intrathecally, topically, or locally. In a further aspect, the invention provides a kit, with instructions, for the prophylactic or therapeutic treatment of a mammal with one or more genetically modified HUCPVC populations, medium conditioned by HUCPVCs (e.g., genetically modified HUCPVCs), and/or compositions that include the soluble fraction of medium conditioned by HUCPVCs (e.g., genetically modified HUCPVCs).

### Human Umbilical Cord Perivascular Cells (HUCPVCs)

Human umbilical cord perivascular cells (HUCPVCs) are a non-hematopoietic, mesenchymal, population of multipotent cells obtained from the perivascular region within the Wharton's Jelly of human umbilical cord (see, e.g., Sarugaser et al., “Human umbilical cord perivascular (HUCPV) cells: A source of mesenchymal progenitors,” Stem Cells 23:220-229 (2005), which is incorporated herein by reference in its entirety). U.S. Patent Application Publication 2005/0148074, International Patent Application Publication WO 2007/128115, International Patent Application Publication WO 2009/129616, and U.S. Pat. Nos. 7,547,546 and 8,481,311 describe methods for the isolation and in vitro culture of HUCPVCs, and are each incorporated by reference herein in their entirety. HUCPVCs are further characterized by relatively rapid proliferation, exhibiting a doubling time, in each of passages 2-7, of about 20 hours (serum dependent) when cultured under standard adherent conditions. Phenotypically, HUCPVCs are characterized, at harvest, as Oct 4⁻, CD14⁻, CD19⁻, CD34⁻, CD44⁺, CD45⁻, CD49e⁺, CD90⁺, CD105(SH2)⁺, CD73(SH3)⁺, CD79b⁻, HLA-G⁻, CXCR4⁺, and c-kit⁺. In addition, HUCPVCs are positive for CK8, CK18, CK19, PD-L2, CD146 and 3G5 (a pericyte marker), at levels higher relative to cell populations extracted from Wharton's jelly sources other than the perivascular region.

When used to recombinantly express a wound healing agent, genetically modified HUCPVCs offer several advantages over other cell-based therapies. Because HUCPVCs exhibit low immunogenicity when administered to an allogeneic or xenogeneic host, they have an increased longevity within the host relative to other allogeneic or xenogeneic cells. HUCPVCs also have established gene expression modalities that result in therapeutically significant levels of a protein or oligonucleotide of interest (e.g., a recombinant polypeptide or oligonucleotide that the HUCPVC has been genetically modified to express, such as a wound healing agent). In addition, although HUCPVCs proliferate rapidly, they have a reduced risk of proliferative disorders relative to other cell-based gene therapy vehicles. Each of these advantageous properties of genetically modified HUCPVCs for the prophylaxis or treatment of a subject (e.g., a human) is discussed in detail below.

The low immunogenicity of genetically modified HUCPVCs make them ideal vehicles for administration to vertebrate subjects, e.g., mammals, such as humans, and particularly to allogeneic or xenogeneic recipients. HUCPVCs have been shown to have low immunogenicity based on their ability avoid detection by the host immune system (see, e.g., Sarugaser et al., supra and U.S. Patent Application Publication 2005/0148074). As such, HUCPVCs harvested from, e.g., a human (i.e., a donor) may be cultured in vitro and administered to another, un-related and HLA-mismatched, human (i.e., a host) without eliciting an allo-specific immune response in the host against the genetically modified HUCPVCs (see, e.g., Ennis et al., “In vitro immunologic properties of human umbilical cord perivascular cells,” Cytotherapy 10(2):174-181 (2008)). Therefore, genetically modified HUCPVCs can be administered to heterologous human populations, or even to xenogeneic populations, without a loss of therapeutic efficacy due to activation of the host immune system. Furthermore, the ability to use HUCPVCs in virtually any vertebrate (e.g., a mammal, such as a human) allows for the large-scale preparation and storage (i.e., “stockpiling”), for example, for use during emergency situations.

The low immunogenicity of HUCPVCs results in increased longevity of these cells in vivo in the treated host subject relative to other allogeneic or xenogeneic cells. Similar mesenchymal cells have been documented to persist in a human host for years when delivered allogeneically (Le Blanc et al., “Fetal mesenchymal stem-cell engraftment in bone after in utero transplantation in a patient with severe osteogenesis imperfecta,” Transplantation 79(11):1607-1614 (2005)), and thus, it can be expected that HUCPVCs will persist within a vertebrate (e.g., a mammalian, such as a human) host for at least weeks to months (e.g., 2 weeks, 3 weeks, 4 weeks, 5 weeks, 6 weeks, 2 months, or more) following injection. The longevity of HUCPVCs used to provide polypeptides or oligonucleotides for therapy or prophylaxis (e.g., by providing a wound healing agent polypeptide or oligonucleotide) offers benefits over other techniques of therapy. Whereas traditional therapeutics typically require multiple administrations to confer a protective or therapeutic effect in an individual, a therapeutically-effective amount of genetically modified HUCPVCs can be administered to an individual in a single dose. Alternatively, two or more doses of the genetically modified HUCPVCs can be administered to provide prophylaxis or therapy.

Another advantageous property of HUCPVCs is that they can be readily genetically modified by a number of standard transfection, transduction, and/or gene editing techniques to allow for the recombinant expression of a therapeutic polypeptide or oligonucleotide (e.g., a wound healing agent). As described further herein, genetic transfer can be achieved, for example, using viral vectors (e.g., adenoviruses, adeno-associated viruses (AAVs), retroviruses, and lentiviruses) and nucleic acid transfection (e.g., DNA plasmids in combination with liposomes, cationic vehicles, or electroporation).

Unlike many other mesenchymal stem cell populations that typically require the donation of bone marrow, HUCPVCs can be reliably collected from human umbilical cords that are normally discarded following birth. In industrialized nations, human umbilical cord blood products are now routinely collected and stored for possible future self- or allo-transplantation. As such, the collection of HUCPVCs for expansion and genetic modification, according to the methods of the invention, are free of many of the logistical constraints associated with the collection of other mesenchymal stem cell populations.

Finally, HUCPVCs have a short population doubling time (see, e.g., Sarugaser et al., 2005, supra) that allows for the rapid and large-scale preparation of genetically modified HUCPVCs for administration to a subject (e.g., a mammal, such as a human) in need thereof. HUCPVCs substantially lack the enzyme telomerase, and therefore the risk of developing proliferative diseases is minimal as these cells cannot divide more than a prescribed number of divisions before apoptosis occurs. In animal experiments, HUCPVCs are not known to generate tumors, even when administered in numbers orders of magnitude larger than clinically applicable.

### Wound Healing Agents

HUCPVCs can be genetically modified to express one or more wound healing agents. When administered in a therapeutically-effective amount, the genetically modified HUCPVCs can inhibit, reduce, prevent, or treat a wound or a symptom associated with a wound. A wound healing agent may be, for example, a nucleic acid (e.g., an oligonucleotide, such as an siRNA) or a polypeptide. Immunomodulatory oligonucleotides or polypeptides can also be expressed in HUCPVCs to modulate (e.g., increase or decrease) host immune responses. Polypeptides expressed in HUCPVCs can be secreted or displayed on the plasma membrane surface (e.g., a membrane-bound receptor or ligand). One or more (e.g., two, three, four, five, six, seven, eight, nine, ten, or more) wound healing agents can be co-expressed in a single HUCPVC.

In any case where the wound healing agent also has anti-cancer activity, there is the optional proviso that the present claims exclude such wound healing agents that are also anti-cancer agents.

A genetically modified HUCPVC can be genetically engineered to express a wound healing agent selected from the group consisting of an anti-fibrotic factor, an anti-inflammatory factor (e.g., a non-antibody inflammatory factor), a stem cell recruitment factor, an extracellular matrix factor, a cytokine or growth factor, a clotting factor, and an angiogenic factor.

The wound healing agent may be an anti-fibrotic factor. The anti-fibrotic factor may be a TGF-β antagonist (e.g., a TGF-β1 antagonist or a TGF-β2 antagonist). The TGF-β antagonist may be decorin or a fusion protein that includes a wound homing peptide, such as CAR (CARSKNKDC, SEQ ID NO: 1), see Jirvinen and Ruoslahti, supra. The TGF-β antagonist may also be, for example, an anti-TGF-β antibody (e.g., fresolimumab, lerdelimumab, and metelimumab) or an anti-TGF-β oligonucleotide (e.g., trabedersen (an antisense oligonucleotide targeting TGF-β2) or an siRNA targeting TGF-β). The anti-TGF-β antibody may be a monoclonal antibody, a humanized antibody, or a human antibody. The anti-TGF-β antibody may be an antibody fragment.

The anti-fibrotic factor may be cathepsin D, cathepsin E, cathepsin S, cathepsin K, cathepsin L, cathepsin B, cathespin C, cathepsin H, cathespin F, cathepsin G, cathepsin O, cathepsin R, cathepsin V (cathepsin 12), cathepsin W, cathepsin Z (cathepsin X), calpin 1, calpin 2, chondroitinase ABC, chondroitinase AC, pancreatic elastase, elastase-2a, elastase-2b, neutrophil elastase, proteinase-3, endogenous vascular elastase, mast cell chymase, mast cell tryptase, plasmin, thrombin, granzyme B, hyaluronidase, chymopapain, chymotrypsin, legumain, collagenase, matrix metalloproteinases (e.g., MMP-1 (collagenase-1), MMP-9, MMP-7 (matrilysin), MMP-8 (collagenase-2), MMP-13 (collagenase-3), MMP-18 (collagenase-4), MMP-2 (gelatinase a), MMP-9 (gelatinase b), MMP-3 (stromelysin-1), MMP-10 (stromelysin-2), MMP-11 (stromelysin-3), MMP-7 (matrilysin), MMP-26 (matrilysin), MMP-12 (metalloelastase), MMP-14 (MT1-MMP), MMP-15 (MT2-MMP), MMP-16 (MT3-MMP), MMP-17 (MT4-MMP), MMP-24 (MT5-MMP), MMP-25 (MT6-MMP), MMP-19, MMP-20 (enamelysin), MMP-x, MMP-23, MMP-27, and MMP-28 (epilysin)), ADAMTS-1, ADAMTS-2, ADAMTS-3, ADAMTS-4 (aggrecanase-1), ADAMTS-5 (aggrecanase-2), ADAMTS-14, papain, subtilisin, subtilisin A, heparanase, TGF-β receptor antagonists (e.g., TGF-β receptor antibodies), angiotensin inhibitors, angiotensin-converting enzyme inhibitors, angiotensin-II receptor antagonists, cannabinoid receptor, or a combination thereof. Anti-fibrotic factors include, but are not limited to, interleukins, interferons (e.g., interferon gamma), cytokines, chemokines, chemotactic molecules, relaxin, hormones (e.g., progesterone, estrogen, testosterone, growth hormone, thyroid hormone, parathyroid hormone, and the like) or a combination thereof.

The wound healing agent may be an anti-inflammatory factor. Any suitable anti-inflammatory factor may be used in the invention. The anti-inflammatory agent (e.g., an inflammatory cytokine antagonist) may be an antibody (e.g., an anti-TNFα antibody (e.g., infliximab, adalimumab, certolizumab pegol, and golimumab), an anti-IL-6 antibody (e.g., siltuximab, elsilimomab, clazakizumab, sirukumab, and olokizumab), an anti-IL-6 receptor antibody (e.g., tocilizumab and sarilumab) or an anti-IL-10 antibody). The antibody may be a monoclonal antibody, a humanized antibody, or a human antibody. The antibody may be an antibody fragment. The anti-inflammatory factor may also be a non-antibody anti-inflammatory factor, including, for example, an inflammatory cytokine antagonist (e.g., an IL-6 antagonist and/or an IL-8 antagonist) or an anti-microbial factor. The inflammatory cytokine antagonist may be, for example, IL-10, LL-37, or thymosin β4. The anti-microbial factor may be, for example, LL-37 or thymosin β4. A non-antibody anti-inflammatory factor (e.g., an inflammatory cytokine antagonist) may also be a soluble receptor fusion protein, e.g., etanercept.

The wound healing factor may be a stem cell recruitment factor. Genes that encode proteins capable of recruiting endogenous stem cells, particularly epithelial progenitors, to the wound are considered to improve the regenerative capacity of the injured tissue (e.g., skin). Any suitable stem cell recruitment factor may be used in the invention. The stem cell recruitment factor may be, for example, TGF-β3, stromal-cell-derived factor (SDF)-1-α, or thymosin β4.

The wound healing factor may be an extracellular matrix factor. Any suitable extracellular matrix factor may be used in the invention. Non-limiting examples of extracellular matrix factors that may be used in the invention include collagen (e.g., collagen-I, collagen-III, or collagen-VI), fibronectin, vitronectin, laminin, cartilage oligomeric matrix protein (COMP), tenascin-C, tenascin-X, elastin, keratin (e.g., K6, K16), tissue inhibitor of metalloproteinase-1 (TIMP-1), albumin, osteonectin, thrombospondin (e.g., thrombospondin-1 or thrombospondin-2), proteoglycans (e.g., versican, syndecan, glypicans, perlecan, lumican, and heparan sulfate), glycosaminoglycans (e.g., hyaluronan/hyaluronic acid), and integrins.

The wound healing factor may be an angiogenic factor. Any suitable angiogenic factor may be used in the invention. Non-limiting examples of angiogenic factors that may be used in the invention include vascular endothelial growth factors (VEGFs), including, e.g., VEGF-A, VEGF-B, VEGF-C, VEGF-D, VEGF-E, and placental growth factor (PIGF); angiopoetins and angiopoetin-like proteins (e.g., ANGPT1, ANGPT2, ANGPT4, ANGPTL1, ANGPTL2, ANGPTL3, ANGPTL4, ANGPTL5, ANGPTL6, and ANGTPL7); fibroblast growth factors (FGFs), including FGF-1 and FGF-2; epidermal growth factor (EGF); transforming growth factors (TGFs), including TGF-α and TGF-β, tumor necrosis factors (TNFs), including TNF-α; colony stimulating factors (CSFs), including granulocyte macrophage colony-stimulating factor (GM-CSF) and macrophage colony stimulating factor (M-CSF, also known as CSF1); and nitric oxide synthase (NOS).

The wound healing factor may be a clotting factor. Any suitable clotting factor may be used in the invention. The clotting factor may be a full-length, unprocessed clotting factor or a processed or activated clotting factor. Non-limiting examples of clotting factors that may be used in the invention include factor I (fibrinogen/fibrin), factor II (prothrombin), CD142 (also known as tissue factor, tissue thromboplastin, or factor III), factor V, factor VII, factor VIII, factor IX, factor X, factor XI, factor XII, factor XIII, von Willebrand factor, prekallikrein, high-molecular weight kininogen (HMWK), fibronectin, antithrombin III, heparin cofactor II, protein C, protein S, protein Z, plasminogen, tissue plasminogen activator (tPA), and urokinase.

The wound healing factor may be a growth factor or cytokine. Any suitable growth factor or cytokine may be used in the invention. Non-limiting examples of growth factors and cytokines include tumor necrosis factor (TNF), such as TNF-α; interferons (e.g., interferon-α, interferon-β, and interferon-γ); interleukins (e.g., IL-1, IL-β, IL-2, IL-3, IL-4, IL-5, IL-6, IL-7, IL-8, IL-9, IL-10, IL-11, IL-12, IL-13, and IL-14); granulocyte macrophage colony-stimulating factor (GM-CSF); granulocyte colony-stimulating factor (G-CSF); chemokines, including CXC (e.g., CXCL10, IL-8/CXCL8, CXCL1, SDF-1), CC (e.g., CCL3 (MIP-1-α), RANTES (CCL5), and MCP-1), and C family chemokines; members of the transforming growth factor-beta (TGF-β) superfamily, including TGF-β1, TGF-β2, and TGF-β3), platelet derived growth factor (PGDF), including PDGF-AA, PDGF-BB, and PDGF-AB; insulin-like growth factors (IGFs), including IGF-I, IGF-II, and des(1-3)-IGF (brain IGF1); epidermal growth factor (EGF), including heparin binding EGF (HB-EGF); fibroblast growth factors (e.g., acidic FGF (FGF-1), basic FGF (FGF-2), FGF-7, and FGF-10; vascular endothelial growth factors (VEGFs), including VEGF-A, VEGF-B, VEGF-C, VEGF-D, VEGF-E, and PIGF; keratinocyte growth factor (KGF), e.g., KGF-1; bone morphogenetic proteins (BMPs, e.g., BMP-2, BMP-4, BMP-6, and BMP-7); activin; brain-derived neurotrophic factor (BDNF), nerve growth factor (NGF), e.g., NGF-13); neurotrophin-3; connective tissue growth factor (CTGF); erythropoietin (EPO); and thrombopoietin (TPO).

### Genetic Modification of HUCPVCs

Recombinant expression of nucleic acids and polypeptides (e.g., wound healing agents) in HUCPVCs can be accomplished by using several different standard gene transfer modalities. These modalities are discussed further below. Exemplary methods of genetically modifying HUCPVCs are also discussed in International Patent Application Publication WO 2007/128115, herein incorporated by reference.

### Transduction (Viral Vectors)

Transduction is the infection of a target cell (e.g., a HUCPVC) by a virus that promotes genetic modification of the target cell. Many viruses bind and infect mammalian cells and can be used to introduce genetic material (e.g., a donor gene, such as a gene encoding a wound healing agent) into the host cell as part of their replication cycle. In viruses modified for gene transfer, the donor gene (e.g., a gene encoding a wound healing agent) is inserted into the viral genome. Additional modifications may be made to the virus to improve infectivity or tropism (e.g., pseudotyping), to reduce or eliminate replicative competency, and/or to reduce immunogenicity. The newly-introduced donor gene will be expressed in the infected host cell or organism and, if replacing a defective host gene, can ameliorate conditions or diseases caused by the defective gene.

Examples of viral vectors that can be used to deliver genetic material (e.g., a donor gene, such as a gene encoding a wound healing agent) include, but are not limited to, a retrovirus, adenovirus (e.g., Ad2, Ad5, Ad11, Ad12, Ad24, Ad26, Ad34, Ad35, Ad40, Ad48, Ad49, Ad50, and Pan9 (also known as AdC68)), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g. measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus), and poxvirus (e.g., vaccinia, modified vaccinia Ankara (MVA), fowlpox and canarypox). Other viruses useful for delivering polynucleotides encoding a wound healing agent to a HUCPVC include Norwalk virus, togavirus, flavivirus, reoviruses, papovavirus, hepadnavirus, and hepatitis virus. Adenoviruses and retroviruses (including lentiviruses) are particularly attractive modalities for gene therapy applications, as discussed below, due to the ability to genetically modify and exploit the life cycle of these viruses.

Adenoviruses

Recombinant adenoviral vectors offer several significant advantages for the expression of a wound healing agent(s) in HUCPVCs. The viruses can be prepared at extremely high titer, infect non-replicating cells, and confer high-efficiency and high-level transduction of target cells in vivo after directed injection or perfusion. Furthermore, as adenoviruses do not integrate their DNA into the host genome, this gene therapy modality has a reduced risk of inducing spontaneous proliferative disorders. In animal models, adenoviral gene transfer has generally been found to mediate high-level expression for approximately one week. The duration of transgene expression may be prolonged, and ectopic expression reduced, by using tissue-specific promoters. Other improvements in the molecular engineering of the adenoviral vector itself have produced more sustained transgene expression and less inflammation. This is seen with so-called “second generation” vectors harboring specific mutations in additional early adenoviral genes and “gutless” vectors in which virtually all the viral genes are deleted utilizing a cre-lox strategy (Engelhardt et al., Proc. Natl. Acad. Sci. USA 91:6196-6200 (1994) and Kochanek et al., Proc. Natl. Acad. Sci. USA 93:5731-5736 (1996)). Examples of adenoviruses that can be used as a viral vector of the invention include those having, or derived from, the serotypes Ad2, Ad5, Ad11, Ad12, Ad24, Ad26, Ad34, Ad35, Ad40, Ad48, Ad49, Ad50, and Pan9 (also known as AdC68).

Adeno-Associated Viruses

Recombinant adeno-associated viruses (rAAV), which are derived from non-pathogenic parvoviruses, can be used to express a donor gene, such as a gene encoding a wound healing agent(s), as these vectors evoke almost no cellular immune response, and produce transgene expression lasting months in most systems. The AAV genome is built of single stranded DNA, and includes inverted terminal repeats (ITRs) at both ends of the DNA strand, and two open reading frames: rep and cap, encoding replication and capsid proteins, respectively. A donor gene (e.g., a gene encoding a wound healing agent) can replace the native rep and cap genes. AAVs can be made with a variety of different serotype capsids which have varying tropism for different tissue types. Examples of AAV serotypes that can be used include but are not limited to AAV1, AAV2, AAV3, AAV4, AAV5, AAV6, AAV7, AAV8, AV9, and AAVrh10. AAV vectors can be produced, for example, by triple transfection of subconfluent HEK293 cells by three plasmids: AAV cis-plasmid containing the donor gene of interest (e.g., a gene encoding a wound healing agent), AAV trans-plasmid containing AAV rep and cap genes, and an adenovirus helper plasmid, e.g., pDF6. Incorporation of a tissue-specific promoter is, again, typically beneficial.

Retroviruses

Another viral vector that can be used to deliver a wound healing agent(s) into a subject or cells is a retrovirus, including a lentivirus. As opposed to adenoviruses, the genetic material in retroviruses is in the form of RNA molecules, while the genetic material of their hosts is in the form of DNA. When a retrovirus infects a host cell, it will introduce its RNA together with some enzymes into the cell. This RNA molecule from the retrovirus will produce a double-stranded DNA copy (provirus) from its RNA molecules through a process called reverse transcription. Following transport into the cell nucleus, the proviral DNA is integrated in a host chromosome, permanently altering the genome of the infected cell and any progeny cells that may arise. The ability to permanently introduce a gene encoding a polypeptide or oligonucleotide into a cell such as a HUCPVC is the defining characteristic of retroviruses used for gene therapy. Retroviruses include lentiviruses, a family of viruses including human immunodeficiency virus (HIV) that includes several accessory proteins to facilitate viral infection and proviral integration. Additional examples of retroviruses include: avian leukosis-sarcoma, mammalian C-type, B-type viruses, D-type viruses, HTLV-BLV group, and spumavirus (Coffin, J. M., Retroviridae: The viruses and their replication, In Fundamental Virology, Third Edition, B. N. Fields, et al., Eds., Lippincott-Raven Publishers, Philadelphia, 1996).

A retrovirus for gene therapy may be one that is modified to direct the insertion of the donor gene incorporated in the genome of the virus into a non-arbitrary position in the genome of the host, e.g., using a zinc finger nuclease or by including sequences, such as the beta-globin locus control region, to direct the site of integration to specific chromosomal sites. Retroviruses and lentiviruses have considerable utility for gene therapy applications. Current, “third-generation” lentiviral vectors feature total replication incompetence, broad tropism, and increased gene transfer capacity for mammalian cells (see Mangeat, B. and Trono, D., “Lentiviral vectors and antiretroviral intrinsic immunity,” Human Gene Therapy 16(8):913-920 (2005) and Wiznerowicz, M. and Trono, D., “Harnessing HIV for therapy, basic research and biotechnology,” Trends Biotechnol. 23(1):42-7 (2005)). Lentiviruses pseudotyped with, e.g., vesicular stomatitis virus glycoprotein (VSV-G) or feline endogenous virus RD114 envelope glycoprotein can be used to transduce HUCPVCs (see, e.g., Zhang et al., “Transduction of bone-marrow-derived mesenchymal stem cells by using lentivirus vectors pseudotyped with modified RD 114 envelope glycoproteins,” J. Virol. 78(3):1219-1229 (2004)). U.S. Pat. Nos. 5,919,458, 5,994,136, and 7,198,950, hereby incorporated by reference, describe the production and use of lentiviruses to genetically modify target cells.

Other Viral Vectors

Besides adenoviral and retroviral vectors, other viral vectors and techniques are known in the art that can be used to transfer a donor gene encoding a desired polypeptide or oligonucleotide (e.g., a gene encoding a wound healing agent) into a subject or cells. These viruses include, e.g., poxviruses (e.g., vaccinia virus and modified vaccinia virus Ankara (MVA); see, e.g., U.S. Pat. Nos. 4,603,112 and 5,762,938), herpesviruses, togaviruses (e.g., Venezuelan Equine Encephalitis virus; see, e.g., U.S. Pat. No. 5,643,576), picornaviruses (e.g., poliovirus; see, e.g., U.S. Pat. No. 5,639,649), baculoviruses, and others described by Wattanapitayakul and Bauer (Biomed. Pharmacother 54:487-504 (2000)), and citations therein. Other viruses useful for delivering donor genes (e.g., wound healing agents) include Norwalk virus, flavivirus, reoviruses, papovavirus, hepadnavirus, and hepatitis virus, for example.

### Transfection

Naked DNA and Oligonucleotides

Naked DNA or oligonucleotides (e.g., DNA vectors such as plasmids) encoding wound healing agents can also be used to genetically modify HUCPVCs. This is the simplest method of non-viral transfection. Clinical trials carried out using intramuscular injection of a naked DNA plasmid have had some success; however expression has been low in comparison to other methods of transfection. Other efficient methods for delivery of naked DNA exist such as electroporation and the use of a “gene gun,” which shoots DNA-coated gold particles into the cell using high pressure gas.

Lipoplexes and Polyplexes

To improve the delivery of a DNA vector (e.g., a plasmid) into a HUCPVC, the DNA can be protected from damage and its entry into the cell facilitated. Lipoplexes and polyplexes have the ability to protect transfer DNA from undesirable degradation during the transfection process. Plasmid DNA can be covered with lipids in an organized structure like a micelle or a liposome. When the organized structure is complexed with DNA it is called a lipoplex. There are three types of lipids, anionic (negatively-charged), neutral, or cationic (positively-charged). Lipoplexes that utilize cationic lipids have proven utility for gene transfer. Cationic lipids, due to their positive charge, naturally complex with the negatively charged DNA. Also as a result of their charge they interact with the cell membrane, endocytosis of the lipoplex occurs, and the DNA is released into the cytoplasm. The cationic lipids also protect against degradation of the DNA by the cell.

Complexes of polymers with DNA are called polyplexes. Most polyplexes consist of cationic polymers and their production is regulated by ionic interactions. One large difference between the methods of action of polyplexes and lipoplexes is that polyplexes cannot release their DNA load into the cytoplasm, so to this end, co-transfection with endosome-lytic agents (to lyse the endosome that is made during endocytosis) such as inactivated adenovirus must occur. However, this is not always the case; polymers such as polyethylenimine have their own method of endosome disruption as does chitosan and trimethylchitosan.

Gene Editing

Gene editing is another approach that can be used to genetically modify HUCPVCs. Broadly, gene editing approaches are based on precise, targeted changes to the genome of organisms. Gene editing may be used to alter the genome sequence (for example, by incorporation of point mutations, insertions, or deletions). Gene editing approaches can be used to ‘knock-in’ heterologous nucleic acid sequences into the genome at targeted locations. A variety of gene editing approaches are known in the art, including but not limited to clustered regularly interspaced short palindromic repeats (CRISPR)-Cas (e.g., Cas9) gene editing (see, e.g., U.S. Pat. Nos. 8,697,359 and 8,771,945), transcription activator-like effector based nuclease (TALEN) gene editing (see, e.g., Ding et al., Cell Stem Cell 12(2):238-251, (2013)), zinc-finger nuclease (ZFN) gene editing (see, e.g., Urnov et al., Nature Reviews Genetics 11: 636-646, (2010)), or meganuclease gene editing (see, e.g., U.S. Pat. No. 8,021,867).

Dendrimers

Dendrimers may be also be used to genetically modify HUCPVCs. A dendrimer is a highly branched macromolecule with a spherical shape. The surface of the particle may be functionalized in many ways, and many of the properties of the resulting construct are determined by its surface. In particular it is possible to construct a cationic dendrimer (i.e., one with a positive surface charge). When in the presence of genetic material such as a DNA plasmid, charge complementarity leads to a temporary association of the nucleic acid with the cationic dendrimer. On reaching its destination the dendrimer-nucleic acid complex is then taken into the HUCPVC via endocytosis.

Hybrid Methods

Hybrid methods of genetic modification have been developed that combine two or more techniques. Virosomes, for example, combine liposomes with an inactivated virus. This approach has been shown to result in more efficient gene transfer in respiratory epithelial cells than either viral or liposomal methods alone. Other methods involve mixing other viral vectors with cationic lipids or hybridizing viruses. Each of these methods can be used to facilitate transfer of a DNA vector (e.g., a plasmid) into a HUCPVC.

Soluble Factors, Conditioned Medium, and Compositions Thereof

The invention features medium conditioned by HUCPVCs, including genetically modified HUCPVCs, such as HUCPVCs genetically modified to express one or more wound healing agents. The invention also features compositions that include the soluble fraction of medium conditioned by HUCPVCs, including genetically modified HUCPVCs, and subfractions thereof. The conditioned medium or compositions that include the soluble fraction of such conditioned medium may include one or more soluble factors produced and secreted by HUCPVCs, including wound healing agents. The medium conditioned by HUCPVCs may include one or more wound healing agents selected from the group consisting of an anti-fibrotic factor, an anti-inflammatory factor (e.g., a non-antibody inflammatory factor), a stem cell recruitment factor, an extracellular matrix factor, a cytokine or growth factor, a clotting factor, and an angiogenic factor, such as those described herein.

The invention features one or more soluble factors produced by HUCPVCs (e.g., genetically modified HUCPVCs). The one or more soluble factors may include a wound healing agent (e.g., an anti-fibrotic factor, an anti-inflammatory factor (e.g., a non-antibody inflammatory factor), a stem cell recruitment factor, an extracellular matrix factor, a cytokine or growth factor, a clotting factor, and an angiogenic factor, such as those described herein). The one or more soluble factors secreted by HUCPVCs (e.g., genetically modified HUCPVCs expressing a wound healing agent) can be determined, for example, using mass spectrometry (e.g., MALDI-TOF/TOF mass spectrometry, for example, as described by Walter et al. J. Stem Cells Regen. Med. 11(1):18-24, 2015). The one or more soluble factors may include, for example, collagen, (e.g., collagen-I and collagen VI), laminin, cartilage oligomeric matrix protein (COMP), lumican, secreted protein acidic and rich in cysteine (SPARC), insulin like growth factor binding protein 1 (IGFBP-1), heparin sulfate proteoglycan (HSPG), fibronectin, and/or decorin.

The one or more soluble factors (including wound healing agents) may be provided as an extract obtained when HUCPVCs (e.g., genetically modified HUCPVCs) are removed from the medium conditioned by their growth, such as by centrifugation. When centrifugation is employed, the extract is provided as the supernatant. Suitable HUCPVC culturing conditions are exemplified herein. The extract is obtained by separating the cells from the conditioned culturing medium, such as by centrifugation. The soluble factor(s) may also be provided as a wound healing fraction of such extract. An extract fraction having wound healing activity is also useful herein, and can be identified using the wound healing assays described herein (e.g., the scratch assay described in Example 4 and the excisional wound assay described in Example 5). These extract fractions can be obtained by fractionating the HUCPVC extract using any convenient technique, including but not limited to solvent extraction, HPLC fractionation, centrifugation, size exclusion, salt or osmotic gradient fractionation and the like. Eluted or collected fractions can then be subjected to the wound healing assay and fractions active for wound healing can be identified. A fraction with wound healing activity can be used in a method of treating wounds, such as those described herein.

The HUCPVCs may be grown under substantially serum-free conditions. Medium conditioned by HUCPVCs (e.g., genetically modified HUCPVCs) under substantially serum-free conditions, or compositions that include the soluble fraction of such conditioned medium, can be used in any of the methods of prophylaxis or treatment described herein. HUCPVCs can be grown or maintained under substantially serum-free conditions, for example, by culturing HUCPVCs through one or more passages (e.g., one, two, three, four, five, six, seven, eight, nine, ten, or more passages) under substantially serum-free conditions. The HUCPVCs may be grown under substantially serum-free conditions for greater than 48 hours (e.g., 50 hours, 60 hours, 72 hours, or more). CM media from HUCPVCs can be produced, for example, by washing the cells twice with phosphate buffered saline (PBS), followed by incubating the cells in a minimal culture medium (e.g., unsupported THERAPEAK® MSCGM-CD (Lonza), a synthetic interstitial fluid (e.g., AQIX®, or chemically defined transport medium (e.g., ZTM™ (Incell)). Conditioned medium can be prepared by culturing HUCPVCs (e.g., HUCPVCs genetically modified to express a wound healing agent, as described herein) under substantially serum-free conditions for at least 5, 10, 15, 20, 24, 36, or 48 hours or more.

The invention also features extracts produced by lysis of HUCPVCs (including genetically modified HUCPVCs). Any suitable lysis method may be used, including mechanical lysis (e.g., by bead beating or mortar and pestle grinding), sonication, enzymatic lysis, detergent lysis, and the like. Fractionation methods as described above can be used to obtain lysate fractions that have wound healing activity. Extracts produced by lysis of HUCPVCs (including genetically modified HUCPVCs) can be used in any of the methods of prophylaxis or treatment described herein.

### Subjects for Wound Treatment

Subjects that can benefit from the administration of genetically modified HUCPVCs (e.g., HUCPVCs genetically modified to express one or more wound healing agents), medium conditioned by HUCPVCs (e.g., genetically modified HUCPVCs), and/or compositions that include the soluble fraction of such conditioned medium, according to the methods of the invention, to treat, inhibit, reduce, ameliorate, or prevent a wound include vertebrates, such as birds (e.g., poultry such as chickens, turkeys, geese, ducks, grouse, swans, peacocks, pigeons, doves, and pheasants), reptiles (e.g., snakes and lizards), amphibians (e.g., frogs and salamanders), mammals (e.g., humans, non-human primates (e.g., monkeys, chimpanzees, apes), ungulates (e.g., horses, cows, goats, pigs, sheep, donkeys, and deer), dogs, and cats. In particular, the subject is a human. In some instances, the subject may be a neonate, a child, an adolescent, or an adult.

Any suitable wound may be treated by the compositions and methods described herein. Wounds that may be treated include, but are not limited to, open wounds, closed wounds, chronic wounds, and burns. An open wound may be an incision, a laceration, an abrasion, an avulsion, a puncture wound, a penetration wound, or a gunshot wound. A closed wound may be a hematoma or a crush injury. A chronic wound may be a venous ulcer, a diabetic ulcer (e.g., a diabetic foot ulcer), or a pressure ulcer. The compositions and methods of the invention may also be used for prophylaxis or treatment of symptoms associated with wounds or complications arising from wounds. A wound may be in any organ including, for example, skin, kidney, lung, liver, the central nervous system, bone, bone marrow, the cardiovascular system, an endocrine organ, or the gastrointestinal system. In particular, the wound may be a skin wound.

### Formulation, Dosing, and Administration

The invention features genetically modified HUCPVCs that express a therapeutically effective amount of one or more wound healing agents, medium conditioned by HUCPVCs (including genetically modified HUCPVCs; such as medium conditioned by HUCPVCs under substantially serum-free conditions), and compositions that include the soluble fraction of medium conditioned by HUCPVCs (including genetically modified HUCPVCs).

Genetically modified HUCPVCs, medium conditioned by HUCPVCs, and/or compositions that include the soluble fraction of medium conditioned by HUCPVCs can be formulated for parenteral (e.g., intramuscular, sub-cutaneous, and intravenous), intranasal, topical, oral, or local administration, for prophylactic or therapeutic treatment. For example, the compositions can be formulated for transdermal delivery, or by injection, such as by intravenous, intramuscular, or subcutaneous injection or by intraarticular injection at areas affected by the condition (e.g., wound or surrounding areas). Additional routes of administration include intravascular, intra-arterial, intraperitoneal, intraventricular, intraepidural, as well as nasal, ophthalmic, intrascleral, intraorbital, rectal, topical (e.g., as an ointment or salve or as a wound dressing), or aerosol inhalation administration. Administration may be systemic or local.

In prophylactic applications, genetically modified HUCPVCs can be administered to a subject (e.g., a human) with a clinically determined predisposition or increased susceptibility to a wound (e.g., a diabetes patient). The compositions of the invention can be administered to the subject (e.g., a human) in an amount sufficient to delay, reduce, or preferably inhibit the onset of clinical disease or disease symptoms caused by, or resulting in, a wound.

In therapeutic applications, genetically modified HUCPVCs, medium conditioned by HUCPVCs, and/or compositions that include the soluble fraction of such medium can be administered to a subject (e.g., a human) already suffering from a wound to treat or at least partially arrest or ameliorate the symptoms of the wound. The number of HUCPVCs, or the amount of medium conditioned by HUCPVCs or composition that includes the soluble fraction of such medium that is adequate to accomplish this purpose is defined as a “therapeutically effective dose.” Amounts effective for this use may depend on the severity of the disease or condition and the weight and general state of the patient.

The total number of genetically modified HUCPVCs administered to a subject in single or multiple doses according to the methods of the invention can be, for example, about 10¹, about 10², about 10³, about 10⁴, about 10⁵, about 10⁶, about 10⁷, about 10⁸, about 10⁹, or more cells, although an effective dose will typically lie in the range of about 10³ to 10⁷ cells (e.g., about 10³ to 10⁴ cells, about 10³ to 10⁵ cells, about 10³ to 10⁶ cells, about 10³ to 10⁷ cells, about 10⁴ to 10⁵ cells, about 10⁴ to 10⁶ cells, about 10⁴ to 10⁷ cells, about 10⁵ to 10⁶ cells, or about 10⁶ to 10⁷ cells) per dose.

The genetically modified HUCPVCs, medium conditioned by HUCPVCs, and/or compositions that include the soluble fraction of such medium can be administered to the subject in need thereof in a single dose. Genetically modified HUCPVCs, medium conditioned by HUCPVCs, or compositions that include the soluble fraction of such medium can also be applied as an initial dose followed by one or more subsequent administrations at hourly, daily, weekly, monthly, or bimonthly intervals. The total effective dose of genetically modified HUCPVCs, medium conditioned by HUCPVCs, or compositions that include the soluble fraction of such medium administered to a subject as a single dose, either as a bolus or by infusion over a relatively short period of time, or can be administered using a fractionated treatment protocol, in which multiple doses are administered over a more prolonged period of time (e.g., a dose every 4-6, 8-12, 14-16, or 18-24 hours, or every 2-4 days, 1-2 weeks, once a month, or once every two months). Alternatively, continuous intravenous infusions sufficient to maintain therapeutically effective concentrations in the blood are contemplated.

The therapeutically-effective amount of a genetically modified HUCPVC, medium conditioned by HUCPVCs, and/or compositions that include the soluble fraction of such medium to be administered to a subject (e.g., a human) according to the methods of the invention can be determined by a skilled artisan. Factors that can be considered include, e.g., individual differences in the subject's age, weight, and/or condition (e.g., the type of wound).

The invention also features the co-administration of an additional (e.g., two or more) genetically modified HUCPVC population to a subject (e.g., a human), in which the additional HUCPVC population expresses one or more different polypeptides or oligonucleotides (e.g., wound healing agent(s)) for prophylactic or therapeutic applications. In some instances, more than two (e.g., three, four, five, six, seven, eight, nine, ten, or more) differently genetically modified HUCPVC populations, each expressing one or more polypeptides or oligonucleotides (e.g., wound healing agent(s)) can be co-administered to a subject for prophylactic or therapeutic applications. For example, cocktails of differently genetically modified HUCPVCs expressing different wound healing agents can be administered to a subject (e.g., a human) to provide multiple wound healing agents, which may be tailored to the nature of the disorder (e.g., the wound).

Alternatively, or in addition, one or more mesenchymal stem cells (MSC) that are not HUCPVCs can be administered. In this case, the MSC can be genetically modified to express a polypeptide or oligonucleotide (e.g., a wound healing agent). It is not always necessary, however, to administer both HUCPVC and MSC populations at the same time or in the same way.

In some cases, the administration of the second population of cells may begin shortly after the completion of the administration period for the first population or vice versa. Such time gap between the two administration periods may vary from one day to one week, to one month, or more. In some cases, two genetically modified HUCPVC populations can be co-administered initially, and subsequently administered singly in following periods (e.g., the administration of two or more HUCPVC populations that individually express a single wound healing agent, e.g., decorin and IL-10). In addition, HUCPVC populations can be modified to express more than one polypeptide or oligonucleotide (e.g., wound healing agents) for prophylactic or therapeutic applications, thus removing the need for multiple administrations.

Single or multiple (e.g., 2, 3, 4, 5, 6, 7, 8, 9, 10, 15, 20, or more) administrations of the compositions of the invention that include an effective amount can be carried out with dose levels and pattern being selected by the treating clinician (e.g., a physician or veterinarian). The dose and administration schedule can be determined and adjusted based on the severity of the wound or likelihood of exposure to, for example, an infectious microbe or a chemical agent. Furthermore, a subject (e.g., a mammal, such as a human) administered genetically modified HUCPVCs can be monitored throughout the course of treatment according to the methods commonly practiced by clinicians or those described herein.

Genetically modified HUCPVCs may be administered in combination with medium conditioned by HUCPVCs (e.g., genetically modified HUCPVCs), compositions that include the soluble fraction of such medium, or both.

One or more physiologically acceptable excipients, diluents, or carriers can also be included in the compositions for proper formulation. Suitable formulations for use in the present invention are found in Remington's Pharmaceutical Sciences, Mack Publishing Company, Philadelphia, Pa., 17th ed., 1985. For a brief review of methods for drug delivery, see, Langer, Science 249:1527-1533, 1990. The medium conditioned by HUCPVCs (e.g., soluble HUCPVCs) may in some cases be the carrier. In the alternative, the medium can be dried, to retain the soluble factor(s) secreted by HUCPVCs (e.g., genetically modified HUCPVCs) and reconstituted in a different vehicle, such as phosphate buffered saline (PBS).

A composition of the invention (e.g., a genetically modified HUCPVC, medium conditioned by HUCPVCs (e.g., genetically modified HUCPVCs), a composition that includes the soluble fraction of such medium, or a pharmaceutical composition thereof) may further include one or more additional therapeutic agents. The additional therapeutic agent may be, for example, an anti-fibrotic factor, an anti-inflammatory factor (e.g., a non-antibody inflammatory factor), a stem cell recruitment factor, an extracellular matrix factor, a cytokine or growth factor, a clotting factor, and an angiogenic factor, such as those described herein. In some instances, the additional therapeutic agent may include an anti-microbial agent, an anti-inflammatory compound, an analgesic, and/or an immunosuppressant, such as those described herein.

A composition of the invention (e.g., a genetically modified HUCPVC, medium conditioned by HUCPVCs (e.g., genetically modified HUCPVCs), and/or a composition that includes the soluble fraction of such medium) may be formulated, for example, as an ointment, salve, lotion, or cream for topical administration to a wound. Such a formulation may include any suitable pharmaceutically acceptable carrier, diluent, or excipient. Suitable carriers include, but are not limited to, mineral oil, liquid petroleum, white petroleum, propylene glycol, emulsifying wax, water, and the like. The formulation may include an additional therapeutic agent, for example, an anti-fibrotic factor, an anti-inflammatory factor (e.g., a non-antibody inflammatory factor), a stem cell recruitment factor, an extracellular matrix factor, a cytokine or growth factor, a clotting factor, and an angiogenic factor, such as those described herein. The additional therapeutic agent may include, for example, an anti-microbial agent, an anti-inflammatory compound, an analgesic, and/or an immunosuppressant, such as those described herein.

A composition of the invention (e.g., a genetically modified HUCPVC (e.g., genetically modified to express one or more wound healing agents), medium conditioned by HUCPVCs (e.g., genetically modified HUCPVCs), and/or a composition that includes the soluble fraction of such medium) may be formulated as a wound dressing (e.g., a transparent film dressing (e.g., TEGADERM™), a hydrocolloid dressing, a hydrofiber dressing (e.g., a carboxymethylcellulose dressing), a hydrogel dressing, an alginate dressing, a collagen dressing, a gauze dressing, a foam dressing, tape, binders, bandages, and combinations thereof. The wound dressing may include an additional therapeutic agent, for example, an anti-fibrotic factor, an anti-inflammatory factor (e.g., a non-antibody inflammatory factor), a stem cell recruitment factor, an extracellular matrix factor, a cytokine or growth factor, a clotting factor, and an angiogenic factor, such as those described herein. The additional therapeutic agent may include, for example, an anti-microbial agent, an anti-inflammatory compound, an analgesic, and/or an immunosuppressant, such as those described herein.

### Additional Therapeutic Regimens

The invention provides for the co-administration of one or more therapeutic agents in combination with genetically modified, medium conditioned by HUCPVCs, or compositions that include the soluble fraction of such medium. For example, an additional therapeutic agent may be administered with genetically modified HUCPVCs described herein at concentrations known to be effective for such therapeutic agents.

The genetically modified HUCPVCs, medium conditioned by HUCPVCs, or compositions that include the soluble fraction of such medium and the additional therapeutic agents can be administered at least one hour, two hours, four hours, six hours, 10 hours, 12 hours, 18 hours, 24 hours, three days, seven days, fourteen days, or one month apart. The dosage and frequency of administration of each component can be controlled independently. The additional therapeutic agents described herein may be admixed with additional active or inert ingredients, e.g., in conventional pharmaceutically acceptable carriers. A pharmaceutical carrier can be any compatible, non-toxic substance suitable for the administration of the compositions of the present invention to a subject. Pharmaceutically acceptable carriers include, for example, water, saline, buffers and other compounds, described, for example, in the Merck Index, Merck & Co., Rahway, N.J. A slow release formulation or a slow release apparatus may be also be used for continuous administration. The additional therapeutic regimen may involve other therapies, including modification to the lifestyle of the subject being treated, administration of wound dressings, and the like.

The additional therapeutic agent can comprise cells. Suitable cells include, without limitation, mesenchymal stem cells, pluripotent stem cells, embryonic stem cells, periosteal cells, osteoprogenitor cells, osteoblasts, osteoclasts, bone marrow-derived cell lines, or any combination thereof.

A composition of the invention may be co-administered with an anti-fibrotic factor. Any anti-fibrotic factor described herein may be used. A composition of the invention may be co-administered with an anti-inflammatory factor. Any anti-inflammatory factor described herein may be used. A composition of the invention may be co-administered with a stem cell recruitment factor. Any of the stem cell recruitment factors described herein may be used. A composition of the invention may be co-administered with an extracellular matrix factor. Any of the extracellular matrix factors described herein may be used. A composition of the invention may be administered with an growth factor or cytokine. Any of the growth factors or cytokines described herein may be used. A composition of the invention may be administered with a clotting factor. Any of the clotting factors described herein may be used. A composition of the invention may be administered with an angiogenic factor. Any of the angiogenic factors described herein may be used.

A composition of the invention may also be co-administered with an anti-microbial agent, an anti-inflammatory compound, an analgesic, or an immunosuppressant.

Anti-Microbial Agents

A composition of the invention (e.g., genetically modified HUCPVC, medium conditioned by HUCPVCs, or compositions that include the soluble fraction of such medium) may be co-administered with an anti-microbial agent. An anti-microbial agent may be, for example, an anti-bacterial agent, an anti-viral agent, an anti-fungal agent, or an anti-protozoal agent.

Exemplary anti-bacterial agents include, for example, aminoglycosides, ansamycins, carbacephems, carbapenems, cephalosporins, glycopeptides, lincosamides, lipopeptides, macrolides, monobactams, nitrofurans, oxazolidinones, penicillins, quinolones, sulfonamides, and tetracyclines. Particular examples of anti-bacterial agents include but are not limited to amifioxacin, amikacin, amoxycillin, ampicillin, aspoxicillin, azidocillin, azithromycin, aztreonam, balofloxacin, benzylpenicillin, biapenem, brodimoprim, cefaclor, cefadroxil, cefatrizine, cefcapene, cefdinir, cefetamet, ceftmetazole, cefoxitin, cefprozil, cefroxadine, ceftarolin, ceftazidime, ceftibuten, ceftobiprole, cefuroxime, cephalexin, cephalonium, cephaloridine, cephamandole, cephazolin, cephradine, chlorquinaldol, chlortetracycline, ciclacillin, cinoxacin, ciprofloxacin, clarithromycin, clavulanic acid, clindamycin, clofazimine, cloxacillin, colistin, danofloxacin, dapsone, daptomycin, demeclocycline, dicloxacillin, difloxacin, doripenem, doxycycline, enoxacin, enrofloxacin, erythromycin, fleroxacin, flomoxef, flucloxacillin, flumequine, fosfomycin, gentamycin, isoniazid, imipenem, kanamycin, levofloxacin, linezolid, mandelic acid, mecillinam, meropenem, metronidazole, minocycline, moxalactam, mupirocin, nadifloxacin, nalidixic acid, netilmycin, netromycin, nifuirtoinol, nitrofurantoin, nitroxoline, norfloxacin, ofloxacin, oxytetracycline, panipenem, pefloxacin, phenoxymethylpenicillin, pivampicillin, pivmecillinam, prulifloxacin, rufloxacin, sparfloxacin, sulbactam, sulfabenzamide, sulfacytine, sulfametopyrazine, sulphacetamide, sulphadiazine, sulphadimidine, sulphamethizole, sulphamethoxazole, sulphanilamide, sulphasomidine, sulphathiazole, teicoplanin, temafioxacin, tetracycline, tetroxoprim, tigecyclin, tinidazole, tobramycin, tosufloxacin, trimethoprim, vancomycin, and pharmaceutically acceptable salts or esters thereof.

Exemplary anti-viral agents include, but are not limited to, acyclovir, brivudine, cidofovir, curcumin, desciclovir, 1-docosanol, edoxudine, famcyclovir, fiacitabine, ibacitabine, imiquimod, lamivudine, penciclovir, valacyclovir, valganciclovir, and pharmaceutically acceptable salts or esters thereof.

Exemplary anti-fungal agents include, but are not limited to, 5-flucytosin, aminocandin, amphotericin B, anidulafungin, bifonazole, butoconazole, caspofungin, chlordantoin, chlorphenesin, ciclopirox olamine, clotrimazole, eberconazole, econazole, fluconazole, flutrimazole, isavuconazole, isoconazole, itraconazole, ketoconazole, micafungin, miconazole, nifuroxime, posaconazole, ravuconazole, tioconazole, terconazole, undecenoic acid, and pharmaceutically acceptable salts or esters thereof.

Exemplary anti-protozoal agents include, but are not limited to, acetarsol, azanidazole, chloroquine, metronidazole, nifuratel, nimorazole, omidazole, propenidazole, secnidazole, sineflngin, tenonitrozole, temidazole, tinidazole, and pharmaceutically acceptable salts or esters thereof.

Anti-Inflammatory Compounds

Anti-inflammatory compounds can be used as an additional therapeutic compound in combination with a composition of the invention. Exemplary anti-inflammatory compounds that may be used in the invention as additional therapeutic agents include, but are not limited to, allopurinol, benzydamine hydrochloride, benzindopyrine hydrochloride, diclofenac, statins, sulindac, sulfasalazine, naroxyn, indomethacin, ibuprofen, flurbiprofen, ketoprofen, aclofenac, aloxiprin, aproxen, aspirin, diflunisal, fenoprofen, mefenamic acid, naproxen, phenylbutazone, piroxicam, meloxicam, salicylamide, salicylic acid, desoxysulindac, tenoxicam, ketoralac, clonidine, flufenisal, salsalate, triethanolamine salicylate, aminopyrine, antipyrine, oxyphenbutazone, apazone, cintazone, flufenamic acid, clonixeril, clonixin, meclofenamic acid, flunixin, colchicine, demecolcine, oxypurinol, dimefadane, indoxole, intrazole, mimbane hydrochloride, paranylene hydrochloride, tetrydamine, fluprofen, ibufenac, naproxol, fenbufen, cinchophen, diflumidone sodium, fenamole, flutiazin, metazamide, letimide hydrochloride, nexeridine hydrochloride, octazamide, molinazole, neocinchophen, nimazole, proxazole citrate, tesicam, tesimide, tolmetin, triflumidate, fenamates (mefenamic acid, meclofenamic acid), nabumetone, celecoxib, etodolac, nimesulide, apazone, gold, tepoxalin; dithiocarbamate, or a combination thereof.

Anti-inflammatory compounds also include other compounds such as steroids, for example, fluocinolone, cortisol, cortisone, hydrocortisone, fludrocortisone, prednisone, prednisolone, methylprednisolone, triamcinolone, betamethasone, dexamethasone, beclomethasone, fluticasone interleukin-1 receptor antagonists, thalidomide (a TNF-α release inhibitor), thalidomide analogues (which reduce TNF-α production by macrophages), quinapril (an inhibitor of angiotensin II, which upregulates TNF-α), aurin-tricarboxylic acid (which inhibits TNF-α), guanidinoethyldisulfide, or a combination thereof.

Cytokines and Growth Factors

Cytokines and growth factors can be used as additional therapeutic agents in combination with a composition of the invention. Exemplary cytokines and growth factors that may be used as an additional therapeutic agent include but are not limited to tumor necrosis factors (TNFs), such as TNF-α; interferons (e.g., interferon-α, interferon-β, and interferon-γ); interleukins (e.g., IL-1, IL-β, IL-2, IL-3, IL-4, IL-5, IL-6, IL-7, IL-8, IL-9, IL-10, IL-11, IL-12, IL-13, and IL-14); granulocyte macrophage colony-stimulating factor (GM-CSF); granulocyte colony-stimulating factor (G-CSF); chemokines, including CXC (e.g., CXCL10, IL-8 (CXCL8), CXCL1, and SDF-1), CC (e.g., CCL3 (MIP-1-α), RANTES (CCL5), and MCP-1), and C family chemokines; members of the transforming growth factor-beta (TGF-β) superfamily, including TGF-β1, TGF-β2, and TGF-β3), platelet derived growth factor (PGDF), including PDGF-AA, PDGF-BB, and PDGF-AB; insulin-like growth factors (IGFs), including IGF-I, IGF-II, and des(1-3)-IGF (brain IGF1); epidermal growth factor (EGF), including heparin binding EGF (HB-EGF); fibroblast growth factors (e.g., acidic FGF (FGF-1), basic FGF (FGF-2), FGF-7, and FGF-10; vascular endothelial growth factors (VEGFs), including VEGF-A, VEGF-B, VEGF-C, VEGF-D, VEGF-E, and PIGF; keratinocyte growth factor (KGF), e.g., KGF-1; bone morphogenetic proteins (BMPs, e.g., BMP-2, BMP-4, BMP-6, and BMP-7); activin; brain-derived neurotrophic factor (BDNF), nerve growth factor (NGF), e.g., NGF-β); neurotrophin-3; connective tissue growth factor (CTGF); erythropoietin (EPO); and thrombopoietin (TPO).

Analgesics

It may be desirable to treat pain associated with a wound of a treated individual. Exemplary analgesics that may be used in the invention include but are not limited to aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine.

Immunosuppressants

It may be desirable to suppress the immune system of a treated individual to prevent e.g., immunopathology associated with an infection or to reduce inflammation associated with a wound. Immunosuppressants can also be used to decrease host rejection of administered HUCPVCs, thereby increasing the longevity of these cells in vivo. Exemplary immunosuppressants include, without limitation, abetimus, deforolimus, everolimus, gusperimus, pimecrolimus, sirolimus, tacrolimus, temsirolimus, anakinra, azathioprine, ciclosporin, leflunomide, methotrexate, mycophenolic acid, and thalidomide.

Additionally, many monoclonal antibodies that cause immunosuppression are also known in the art, including TNF inhibitors (e.g., anti-TNF-α antibodies such as infliximab, adalimumab, and certolizumab pegol), alemtuzumab, afelimomab, aselizumab, atlizumab, atorolimumab, basiliximab, belimumab, bertilimumab, cedelizumab, clenoliximab, daclizumab, dorlimomab aritox, dorlixizumab, eculizumab, efalizumab, elsilimomab, erlizumab, faralimomab, fontolizumab, galiximab, gantenerumab, gavilimomab, golimumab, gomiliximab, ibalizumab, inolimomab, ipilimumab, keliximab, lebrilizumab, lerdelimumab, lumiliximab, maslimomab, mepolizumab, metelimumab, morolimumab, muromonab-CD3, natalizumab, nerelimomab, ocrelizumab, odulimomab, omalizumab, otelixizumab, pascolizumab, pexelizumab, rituxumab, reslizumab, rovelizumab, ruplizumab, siplizumab, talizumab, telimomab aritox, teneliximab, teplizumab, tocilizumab, toralizumab, vapaliximab, vepalimomab, visilizumab, zanolimumab, ziralimumab, and zolimomab aritox.

### EXAMPLES

The following examples are to illustrate the invention. They are not meant to limit the invention in any way.

### Example 1: Genetic Modification of HUCPVCs to Express Elevated Levels of Decorin for Wound Healing

A number of studies have demonstrated that while transplanted mesenchymal stem cells (MSCs) can improve wound healing, they do not eliminate scarring. The limited capacity of MSCs to regenerate the full complement of cell types in normal skin architecture could be improved through a genetic modification approach, in which MSCs are engineered to secrete factors that attract the host's stem cells to re-populate the wound site. In addition, therapeutic MSCs can be engineered to deliver factors known to reduce inflammation, protect against infection, minimize scarring, and promote angiogenesis and proper extracellular matrix organization, including the wound healing agents described herein. Genetic engineering of MSCs to express specific genes encoding, for example, growth factors or cytokines would allow the MSCs to deliver sustained, therapeutic levels of regenerative factors, thereby enhancing the capacity of MSCs to improve wound healing. The expected physiological outcomes of MSC-mediated gene therapy include reducing inflammation to accelerate healing and minimize scarring, and the recruitment and activation of endogenous stem cells from uninjured skin to the wound to regenerate normal skin architecture, composition and function. Genetically modified MSC therapy, which couples the innate healing power of applied MSCs with the targeted delivery of factors known to minimize scar formation and promote skin regeneration, is a promising approach for full regeneration of severe skin wounds.

Genes that encode factors with anti-scarring effects typically have roles in counteracting inflammation. Rapid resolution of the inflammatory phase accelerates wound closure and minimizes the natural over-production of extracellular matrix molecules used to contract the wound, which later gives rise to scar tissue. For example, decorin (Dcn) is a small, naturally occurring extracellular matrix proteoglycan that is associated with collagen fibrils in all connective tissues, and is required for the proper assembly of collagenous matrices. Initiation of Dcn expression is delayed in burn patients, and scar tissue from these patients exhibit an abnormally low amount of Dcn compared with normal skin. The lack of Dcn accounts for the poor organization of collagen fibrils typically associated with scar tissue. These effects can be attributed, at least in part, to Dcn's ability to modulate TGF-β signaling. Many studies have demonstrated the anti-fibrotic effects of Dcn during wound healing in various contexts. Recent reports show that direct injection of recombinant human Dcn can efficiently prevent fibrosis and enhance tissue regeneration, and that Dcn gene transfer promotes muscle regeneration. Despite its success in animal models, attempts to develop a Dcn-based therapy for clinical applications have been unsuccessful, due to the manufacturing challenges of producing active Dcn in vitro, and failure to administer sufficiently large enough quantities for an appropriate duration to elicit an effect.

Therefore, delivery of Dcn and/or other wound healing agents described herein to the wound by MSC-mediated gene transfer poses a promising solution to the current clinical roadblock. The approach of the present invention, at least in part, is to genetically modify human umbilical cord perivascular cells (HUCPVCs) for gene transfer of wound healing agents such as decorin. HUCPVCs exhibit many potent anti-inflammatory and wound healing benefits, and have been shown to be highly efficient vectors for gene transfer.

Previous studies have suggested that MSCs may naturally secrete Dcn, and that this may contribute to their wound healing efficacy in vivo. Consistent with these reports, we have verified by microarray (mRNA; see Example 2) and Western blot (protein; Example 3) analyses that, indeed, HUCPVCs naturally secrete Dcn in culture. We have further established that HUCPVCs, engineered by adenoviral-mediated gene transfer with an episomal human Dcn transgene, secrete higher levels of Dcn, and the level of expression is dependent on the number of transgene copies per cell (see Example 3). We have obtained similar results using a transgene that codes for Dcn fused to a CAR peptide (CAR-Dcn), which homes the Dcn molecule to the vasculature, thereby improving its wound healing efficacy (Järvinen and Ruoslahti, supra) (see Example 3). As described below in Example 3, recombinant Dcn typically exhibits heterogeneity in chondroitin sulfate chains, which produces a smear of Dcn protein on western blots. This heterogeneity is an impediment to approval of recombinant Dcn for use in humans. In contrast, both the endogenous and exogenous Dcn produced by HUCPVCs produces a sharp band on Western blots, indicating that it is a homogeneous protein pool suitable for commercialization. These findings have been validated using two different antibodies targeted against different regions of the Dcn protein. Additionally, the decorin secreted by HUCPVCs is functional and has effects in an in vitro wound healing model (Example 4). Genetically modified HUCPVCs are also tested in various animal wound healing models, including the excisional wound model described in Example 5.

In summary, genetically modified HUCPVCs expressing wound healing agents such as decorin, as well as conditioned medium or the soluble fraction of such medium represent a promising approach for treatment of wounds such as skin wounds (e.g., burns).

### Example 2: Genomic Profiling of HUCPVCs

Materials and Methods

HUCPVCs were cultured in serum- and xeno-free THERAPEAK® MSCGM-CD media (Lonza). Passage 1 (P1) or P2 cells were revived from cryogenic storage and seeded on fibronectin-coated T75 culture vessels at a growth density of 1333 cells per cm². Cells were fed every 3 days. At approximately 80% confluence, cells were imaged by bright field microscopy, then washed with sterile phosphate buffered saline (PBS) and lifted by incubation with TrypLE™ Select. Cells were counted using a Millipore SCEPTER™ cell counter, and reseeded for expansion. The remaining unseeded cells were preserved in RNAprotect® until RNA extraction. Cells were serially cultured, imaged, and RNA harvested until the cells reached senescence (confluence not attained after 6 weeks in passage). mRNA levels in the extracted RNA were interrogated against 14,500 genes using Affymetrix Human Genome U133A 2.0 arrays. Three independent cell lots were cultured and analyzed in parallel. A fourth, independent lot was cultured and analyzed separately.

Results

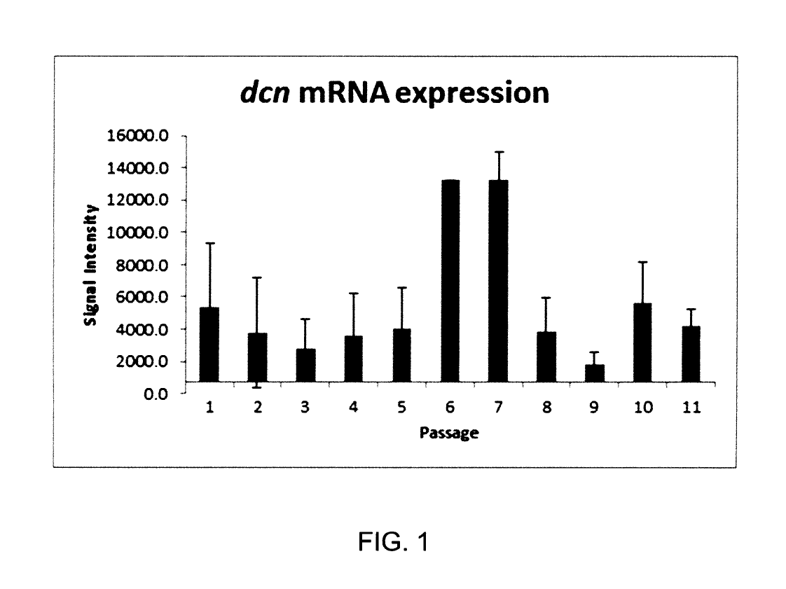

Signal intensities for the decorin (dcn) gene were plotted against average gene expression intensity (720) for the full data set. These data indicate that dcn mRNA was expressed higher than whole-genome average (set as the y intercept) by HUCPVCs in normal culture conditions (FIG. 1).

### Example 3: Genetically Modified HUCPVCs Expressing Decorin

To determine how much Dcn protein is secreted by native HUCPVCs, and whether they can be engineered to secrete higher than endogenous Dcn levels, 100,000 HUCPVCs (Lot 130, P5) were seeded into each well of a 6 well plate. Two constructs were used for genetic engineering: a recombinant adenovirus (pAd5) encoding the full human decorin gene (pAd5-Dcn) and pAd5-CAR-Dcn, which encodes human decorin fused to the CAR peptide that homes to the vasculature and thus can target the fusion protein to wounds (Järvinen and Ruoslahti, supra). A previous study has shown that more tail-vein administered Dcn reached the wound bed when the protein was fused to CAR, thereby improving its efficacy (Järvinen and Ruoslahti, supra).

Both pAd5 constructs used include an internal ribosome entry site (IRES) upstream of an eGFP transgene; this reporter construct produces an eGFP molecule each time a Dcn molecule is produced, and is useful for validating transfection efficiency and transgene expression. The eGFP is not fused to the Dcn protein, but is simply an expression level reporter. Twenty four hours after seeding, cells were incubated for 2 hours with a minimal volume of either media alone (for native cells), or media containing the pA5-Dcn construct at an MOI (multiplicity of infection, the ratio of infective particles to the number of cells) of 20 or 100. These MOIs were selected to initially assess the range in which cells should be engineered to maximize exogenous Dcn expression without toxic effects to the cells. After 2 hours, the virus cocktail was removed and the media replaced. Conditioned media (CM) was collected from the cultured cells and replaced every 72 hours, and stored at −20° C. until analysis.

ELISA Measurement of Dcn in HUCPVC CM

The amount of Dcn present in CM from the native and engineered HUCPVC cells was quantified by enzyme-linked immunosorbent assay (ELISA) (AbCam human ELISA kit, ab99998). Samples were analyzed in duplicate as neat, or diluted to 1/10, 1/100 and 1/1000. Only 1/100 or 1/1000 dilutions were within the linear range of the assay, depending on the sample. A standard curve was plotted, and the amount of Dcn present in each sample extrapolated using absorbance readings within the linear range. The limit of detection for the assay was set at 1.2, or 20% above the absorbance of the lowest standard.

Results

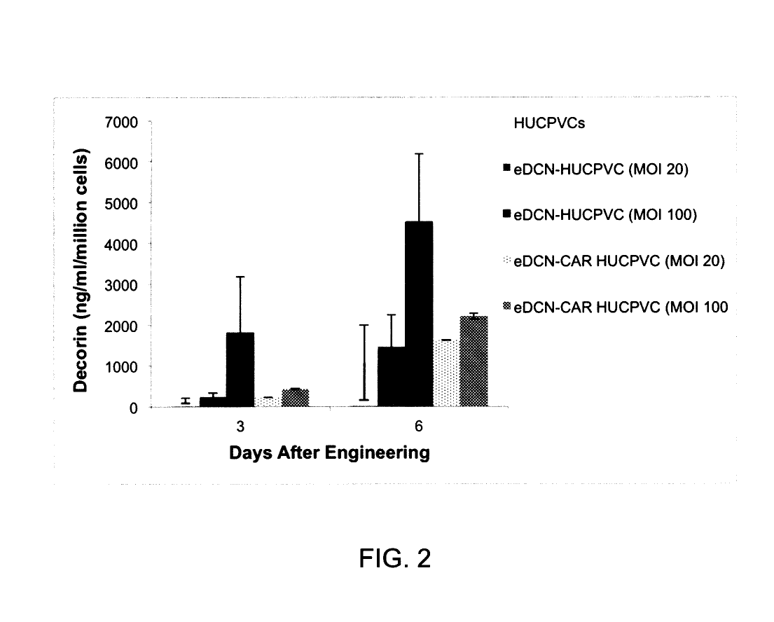

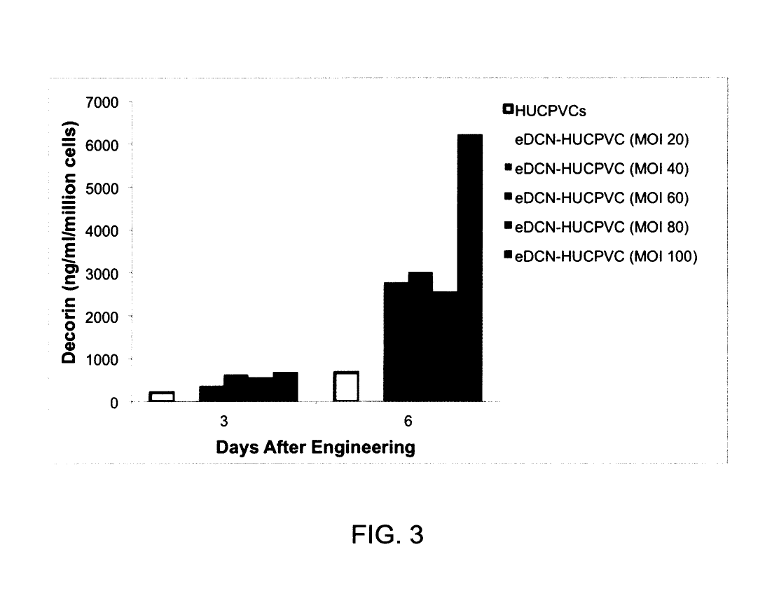

Genetically modified HUCPVCs secreted Dcn and CAR-Dcn into the culture medium (FIG. 2). Dcn was detected in CM from native HUCPVCs, and at significantly higher levels in HUCPVCs genetically modified to express Dcn or CAR-Dcn (FIG. 2). Further, HUCPVCs secrete more decorin as a consequence of higher transgene copy number (FIG. 3).

Twenty four hours after engineering, eGFP was observed in approximately 20% of cells engineered at MOI 100. eGFP accumulated in these cells, as evidenced by increased frequency and intensity of eGFP, and nearly all cells were eGFP positive by day 3. eGFP was extremely faint and barely discernible in cells engineered at MOI 20. Cells engineered at MOI 100 began to exhibit morphological signs of toxicity by day 3 after engineering. By day 7, cells began to detach and dead cells were evident in the culture media. The study was terminated at day 9, as the MOI 100 cultures were too compromised for reliable data analysis.

The amount of Dcn and CAR-Dcn secreted by HUCPVCs was greater on day 6 as compared to day 3 post-engineering (FIG. 2). On day 9, many MOI 100 cells had already begun to detach from the culture vessel and dead cells were evident in the media. Dcn levels present in CM at day 9, though were comparable to Dcn levels in CM observed at day 6. The presence of fewer viable cells at day 9 may be a consequence of eGFP accumulation in these cells or may be related to the very high levels of Dcn secreted by cells engineered with many copies of the Dcn transgene at MOI 100.

The samples analyzed here are 72 hour media collections. The half-life of Dcn has been reported as 2.5 hours in cell culture (Yung et al. “Catabolism of newly synthesized decorin in vitro by human peritoneal mesothelial cells” Perit. Dial. Int. 24(2):147-55, 2004), although its metabolism by HUCPVCs in particular is unknown. Hence, these data may only represent a snapshot of the amount of Dcn in the CM. The quantity of Dcn produced by the cells over a 24 hour period, for example, may in fact be much higher. In addition, the eGFP is produced from the same promoter in the current pAd5 constructs; it is expected that Dcn expression will further increase when dcn is expressed under a dedicated promoter.

Western Blot

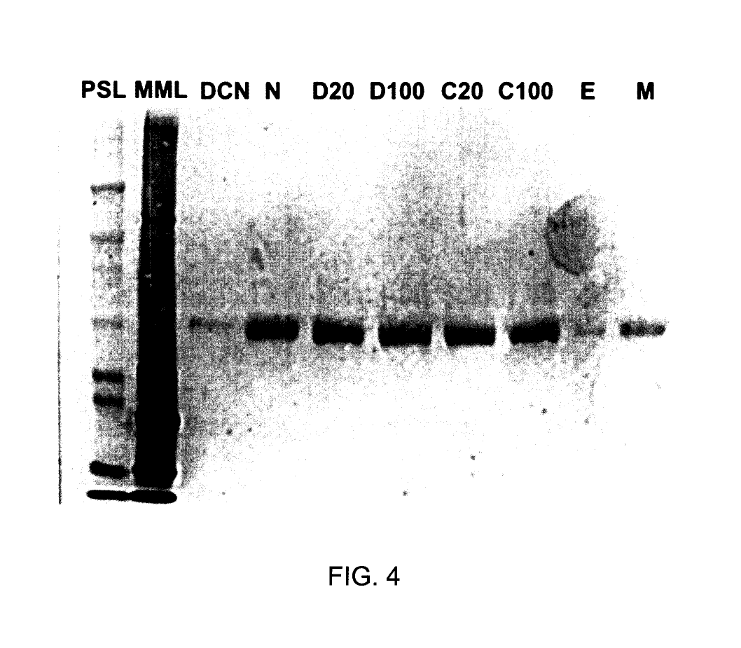

Conditioned medium (CM) samples quantified by ELISA were also analyzed by Western blot. Proteins from conditioned media samples analyzed by ELISA (see above) were diluted 1:10, separated by denaturing sodium dodecyl sulfate (SDS) gel electrophoresis, transferred to a polyvinylidene fluoride (PVDF) membrane, and probed using an anti-human decorin antibody. The blot shown in FIG. 4 represent a dilution series of representative samples. Consistent with the ELISA data, the band intensity from MOI 100 was higher than for MOI 20, and these blots validate the presence of the Dcn protein in CM from native and engineered HUCPVCs, as well as the presence of CAR-Dcn from engineered HUCPVCs. In each of the experimental samples, the Dcn band appears as a sharp band, not a smear. According to literature, smeared bands on a Dcn Western blot are typical for recombinant protein samples, and represent heterogeneity in chondroitin sulfate chains (see Yamaguchi and Ruoslahti, “Expression of human proteoglycan in Chinese hamster ovary cells inhibits cell proliferation” Nature 336(6196):244-246, 1988 and Järvinen and Ruoslahti, supra). This heterogeneity is a current regulatory limitation (Jirvinen and Prince, “Decorin: A Growth Factor Antagonist for Tumor Growth Initiation,” Biomed. Res. Int. 2015:654765, 2015). These observations were validated in a duplicate experiment using an anti-Dcn antibody raised against a different epitope.

### Example 4: Functionality of HUCPVC-Secreted Dcn

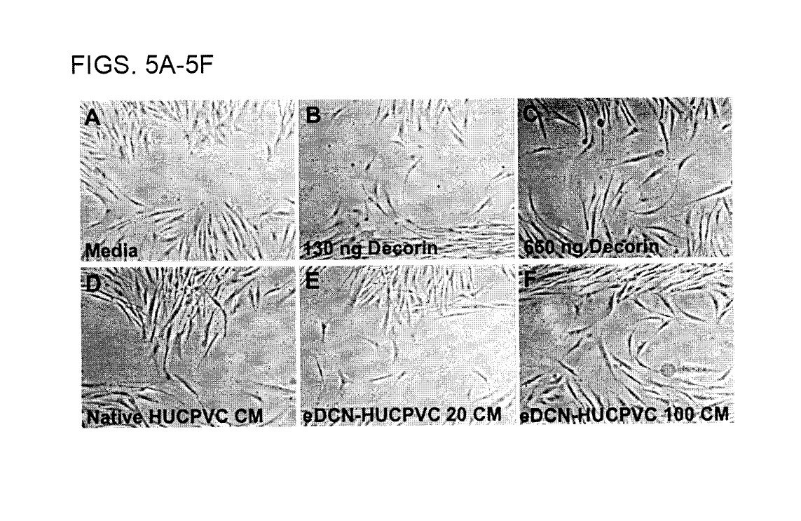

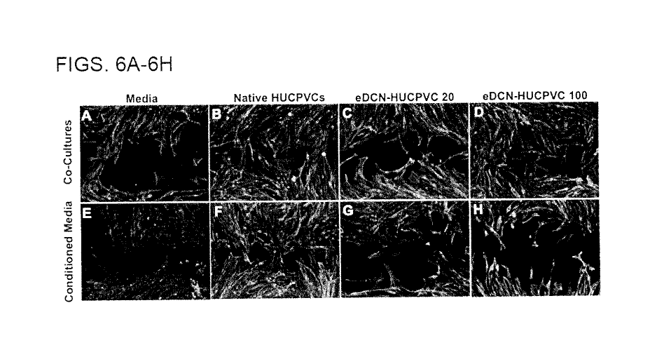

The functionality of HUCPVC-secreted Dcn was tested using an in vitro wound healing model (FIGS. 5A-5F). A monolayer of primary human dermal fibroblasts was “wounded” by applying a scratch through the monolayer, mimicking an open wound. The scratched monolayers were monitored for 18 hours to assess wound closure, i.e., migration of fibroblasts from the wound margin into the gap. Monolayers treated with media alone displayed only modest closure (FIG. 5A). Monolayers treated with media containing a low (130 ng) or high (660 ng) dose of purified Dcn exhibited numerous fibroblasts in the wound, many of which had detached entirely from the monolayer at the wound margin (FIGS. 5B and 5C). Monolayers treated with conditioned media (CM) harvested from unmodified HUCPVCs exhibited nearly complete wound closure (FIG. 5D). Unlike the Dcn-treated scratches, however, the entire wound margins appeared to have converged to close the gap, rather than individual fibroblasts migrating into the scratch. This suggests that both HUCPVCs and Dcn can promote wound re-epithelialization, but through distinct mechanisms. Consistently, monolayers treated with HUCPVCs engineered to secrete either a low (MOI 20) or high (MOI 100) dose of Dcn (see Example 3) showed an intermediate degree of wound closure between the two individual phenotypes (FIGS. 5E and 5F). Some convergence of the wound margin was evident, in addition to the individual fibroblasts populating the gap. Similar trends were observed in monolayers co-cultured with native HUCPVCs, or HUCPVCs engineered to secrete a low or high dose of Dcn (FIGS. 6A-6H).

Dermal fibroblasts are progenitors of myofibroblasts, which exert contractile force on the wound and secrete excessive, disordered collagen that promotes fibrosis. Thus, these in vitro assays indicate that Dcn-engineered HUCPVCs can exert wound closure effects on a highly relevant cell population. Dcn has been shown to directly inhibit the profibrotic factors TGF-β1 and connective tissue growth factor (CTGF) (see Zhao et al., Am. J. Physiol. 277: L412-22 (1999); Zhang et al., Burns 35:527-537 (2009); and Vial et al., J. Biol. Chem. 286:24242-24252 (2011)), which are directly linked to myofibroblast differentiation and recruitment, respectively (see Hinz, J. Invest. Dermatol. 127:526-537 (2007); Liu et al., Arthritis Rheum. 63:239-246 (2011); and Kapoor et al., Fibrogenesis Tissue Repair 1, 3 (2008)). Therefore, without being bound by any theory, it is expected that wounds treated with engineered Dcn-HUCPVCs will contain a reduced myofibroblast population.

Taken together, these assays provide evidence that the exogenous Dcn secreted by engineered HUCPVCs exerts an effect on human dermal fibroblasts in a wound closure context, and that it can modulate the natural wound closure effects of HUCPVCs.

### Example 5: Mouse Wound Healing Model

The effect of genetically modified HUCPVCs expressing Dcn or other wound healing agents described herein, as well as medium conditioned by HUCPVCs (including genetically modified HUCPVCs), or the soluble fraction thereof, can be assessed in an excisional wound-splinting model, for example, as described by Wang et al. “The mouse excisional wound splinting model, including applications for stem cell transplantation,” Nat. Protoc. 8:302-309 (2013) and Shohara et al. “Mesenchymal stromal cells of human umbilical cord Wharton's jelly accelerate wound healing by paracrine mechanisms” Cytotherapy 14(10)1171-81, 2012, which is a well-accepted model for wound healing.

Briefly, BALB/c nude mice (8 week old females) and BALB/c (ICR) mice (8 week old females) can be used for HUCPVC transplantation and conditioned medium injection, respectively. Mice can be anesthetized individually and 6 mm full-thickness excisional wounds were made on the dorsum using a 6 mm tissue punch and Iris scissors. Two wounds can be created, one on each side of the midline of the mouse. A doughnut-shaped splint with a diameter twice that of the wounds can be made from 0.5 mm thick silicone sheet. A fast bonding adhesive, such as Aron ALPHA®, can be used to fix the splint to the skin, followed by interrupted 4-0 nylon sutures to ensure its position.

The wounds can be treated with genetically modified HUCPVCs (including the genetically modified HUCPVCs expressing pAd5-Dcn or pAd5-CAR-DCN described in Example 2) or conditioned medium (CM) produced by the genetically modified HUCPVCs. The cultured HUCPVCs can be detached from culture dishes by treatment with trypsin (0.05% trypsin/ethylenediaminetetraacetic acid (EDTA), and pre-labeled with a fluorescent dye (e.g., PKH26 (Sigma)) according to the manufacturer's instructions. A range of cell dosing can be tested, including a dosing of 1×10⁶ cells, with 0.8×10⁶ cells in 80 μl PBS injected around the wound at four injection sites, and 0.2×10⁶ cells in 20 μl PBS applied directly to the wound bed. For CM injection, 80 μl of CM can be injected around the wound and 20 μl CM was applied to the wound bed. Following the administration of HUCPVCs or CM, a dressing (e.g., TEGADERM™) can be placed over the wound, and the animals can be housed individually.

The wound can be analyzed by digital photography on days 0, 4, 7, 10, and 14 after wounding. The wound can be analyzed by tracing the wound margin using image analysis software (e.g., ImageJ). The percentage of wound closure can be calculated using the following formula: (area of original wound−area of wound at time of analysis)/area of original wound×100.

The wound may also be analyzed by histological analysis, for example, as described by Shohara et al. supra. For example, immunohistochemistry using anti-CD31 antibodies and anti-smooth muscle actin antibodies can be used to visualize capillary density in the wound as a marker of angiogenesis. Additionally, the number of anti-inflammatory M2 macrophages expressing RELM-α, arginase and/or CD11b can be measured by immunohistochemistry. Real time PCR can be performed to determine the expression of selected endogenous wound healing agents, including IL-10, TGF-β (including TGF-β1, TGF-β2, and TGF-β3), VEGF-A, and angiopoietin-1 (ANGPT1).

It is expected that administration of genetically modified HUCPVCs expressing wound healing agents, including the genetically modified HUCPVCs expressing pAd5-Dcn or pAd5-CAR-DCN described in Example 2, will lead to improved wound healing and reduced scarring compared to vehicle controls or non-modified HUCPVCs. It is also expected that administration of conditioned medium produced by these HUCPVCs will exhibit improvements in wound healing and reduction of scarring as compared to vehicle controls or medium conditioned by non-modified HUCPVCs.

### Example 6: Genetically Modified HUCPVCs Expressing an Anti-Inflammatory Factor

Genetically modified HUCPVCs can be engineered to express and secrete an anti-inflammatory factor. For example, HUCPVCs can be engineered to express human IL-10. If desired, HUCPVCs can also be genetically modified to express a second wound healing agent, such as decorin or CAR-decorin.

ELISA experiments using an anti-IL-10 antibody (for example, using a Human IL-10 Quantikine ELISA Kit, R&D systems) can be performed according to the manufacturer's instructions to determine the expression level of IL-10 in medium conditioned by wild-type HUCPVCs or HUCPVCs engineered to express IL-10. Western blot experiments can be used to confirm expression of IL-10.

These HUCPVC cell populations can then be tested in the scratch assay described in Example 4 and in the mouse wound healing model described in Example 5 to assess the effect of genetically modified HUCPVCs expressing IL-10, or IL-10 and decorin, on wound healing. These HUCPVC cell populations can also be administered to a human subject suffering from a wound, e.g., a burn. It is expected that such genetically modified HUCPVCs expressing IL-10 (or IL-10 and decorin) will lead to improved wound healing and reduced scarring compared to controls.

### Example 7: Genetically Modified HUCPVCs Expressing Angiogenic Factors

Genetically modified HUCPVCs can be engineered to express and secrete an angiogenic factor. For example, HUCPVCs can be engineered to express VEGF-A.

ELISA experiments using an anti-VEGF-A antibody (for example, using a Human VEGF Quantikine ELISA Kit, R&D systems) can be performed according to the manufacturer's instructions to determine the expression level of VEGF-A in medium conditioned by wild-type HUCPVCs or HUCPVCs engineered to express VEGF-A. Western blot experiments can be used to confirm expression of VEGF-A.

These HUCPVC cell populations can be tested in the mouse wound healing model described in Example 5 to assess the effect of genetically modified HUCPVCs expressing VEGF-A on wound healing and angiogenesis. These HUCPVC cell populations can also be administered to a human subject suffering from a wound, e.g., a burn. It is expected that such genetically modified HUCPVCs expressing an angiogenic factor, such as VEGF-A, will lead to improved wound healing and angiogenesis compared to controls.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

Other embodiments are within the following claims.

All publications and patent applications mentioned in this specification are herein incorporated by reference to the same extent as if each independent publication or patent application was specifically and individually indicated to be incorporated by reference in their entirety.

1. A human umbilical cord perivascular cell (HUCPVC) which has been genetically modified to express a wound healing agent selected from a non-antibody anti-fibrotic factor, a non-antibody anti-inflammatory factor, a stem cell recruitment factor, and an extracellular matrix factor.

2. The genetically modified HUCPVC of claim 1, wherein the non-antibody anti-fibrotic factor is a transforming growth factor (TGF)-β antagonist.

3. The genetically modified HUCPVC of claim 2, wherein the TGF-β antagonist is decorin.

4. The genetically modified HUCPVC of claim 1, wherein the non-antibody anti-inflammatory factor is an inflammatory cytokine antagonist or an anti-microbial factor.

5. The genetically modified HUCPVC of claim 4, wherein the inflammatory cytokine antagonist is LL-37 or thymosin β4.

6. The genetically modified HUCPVC of claim 4, wherein the anti-microbial factor is LL-37 or thymosin β4.

7. The genetically modified HUCPVC of claim 1, wherein the stem cell recruitment factor is TGF-β3, stromal cell-derived factor (SDF)-1-α, or thymosin β4.

8. The genetically modified HUCPVC of claim 1, wherein the extracellular matrix factor is collagen, laminin, or fibronectin.

9. The genetically modified HUCPVC of any one of claims 1-8, wherein the HUCPVC synthesizes and secretes the wound healing agent.

10. The genetically modified HUCPVC of any one of claims 1-9, wherein the HUCPVC has been genetically modified to express two or more wound-healing agents.

11. The genetically modified HUCPVC of any one of claims 1-10, wherein the HUCPVC has been genetically modified by viral transduction, transfection, dendrimers, gene editing, or a combination thereof.

12. The genetically modified HUCPVC of claim 11, wherein the viral transduction comprises adenoviral transduction, adeno-associated viral (AAV) transduction, or retroviral transduction.

13. The genetically modified HUCPVC of claim 12, wherein the retroviral transduction is lentiviral transduction.

14. The genetically modified HUCPVC of claim 11, wherein the transfection comprises naked nucleic acid transfection, electroporation, gene gun transfection, lipoplex transfection, or polyplex transfection.

15. The genetically modified HUCPVC of claim 11, wherein the gene editing comprises clustered regularly interspaced short palindromic repeats (CRISPR)-Cas gene editing, transcription activator-like effector based nuclease (TALEN) gene editing, zinc-finger nuclease (ZFN) gene editing, or meganuclease gene editing.

16. The genetically modified HUCPVC of any one of claims 1-15, wherein the wound healing agent is endogenous to the HUCPVC.

17. The genetically modified HUCPVC of any one of claims 1-15, wherein the wound healing agent is not endogenous to the HUCPVC.

18. The genetically modified HUCPVC of any one of claims 1-17, wherein the HUCPVCs have a 3G5+, CD45−, CD44+ phenotype.

19. The genetically modified HUCPVC of any one of claims 1-18, wherein the wound healing agent is a wild-type wound healing agent or a variant wound healing agent.

20. The genetically modified HUCPVC of claim 19, wherein the variant wound healing agent is a fusion protein.

21. The genetically modified HUCPVC of claim 20, wherein the fusion protein comprises a fusion partner selected from a targeting moiety and a detectable moiety.

22. The genetically modified HUCPVC of claim 21, wherein the targeting moiety comprises a CAR peptide (CARSKNKDC, SEQ ID NO: 1).

23. The genetically modified HUCPVC of claim 21, wherein the detectable moiety is an epitope tag or a fluorescent protein.

24. A composition comprising the soluble fraction of medium conditioned by the genetically modified HUCPVC of any one of claims 1-23.

25. The composition of claim 24, wherein the composition comprises the wound healing agent.

26. The composition of claim 24 or 25, wherein the composition comprises one or more additional soluble factors produced by the genetically modified HUCPVC.

27. The composition of claim 26, wherein the one or more soluble factors are paracrine factors.

28. The composition of any one of claims 24-27, wherein the HUCPVCs are grown under substantially serum-free conditions.

29. A pharmaceutical composition comprising the genetically modified HUCPVC of any one of claims 1-23 and a pharmaceutically acceptable carrier or excipient.

30. A pharmaceutical composition comprising the composition of any one of claims 24-28 and a pharmaceutically acceptable carrier or excipient.

31. The pharmaceutical composition of claim 29 or 30, further comprising an additional therapeutic agent.

32. The pharmaceutical composition of claim 31, wherein the additional therapeutic agent is selected from an anti-microbial agent, an anti-inflammatory compound, a cytokine or growth factor, an analgesic, or an immunosuppressant.

33. A method of treating a wound in a subject in need thereof, the method comprising administering a therapeutically effective amount of the pharmaceutical composition of any one of claims 29-32 to the subject.

34. A method of treating a wound in a subject in need thereof, the method comprising administering to the subject a therapeutically effective amount of a pharmaceutical composition comprising (i) a genetically modified HUCPVC or (ii) a composition comprising the soluble fraction of medium conditioned by a genetically modified HUCPVC, wherein the HUCPCV has been genetically modified to express a wound healing agent.

35. The method of claim 34, wherein the wound healing agent is selected from the group consisting of an anti-fibrotic factor, an anti-inflammatory factor, a stem cell recruitment factor, an extracellular matrix factor, a cytokine or growth factor, a clotting factor, and an angiogenic factor.

36. The method of claim 35, wherein the anti-fibrotic factor is a TGF-β antagonist.

37. The method of claim 36, wherein the TGF-β antagonist is an anti-TGF-β antibody or a non-antibody TGF-β antagonist.

38. The method of claim 37, wherein the non-antibody TGF-β antagonist is decorin.

39. The method of claim 35, wherein the anti-inflammatory factor is an inflammatory cytokine antagonist or an anti-microbial factor.

40. The method of claim 39, wherein the inflammatory cytokine antagonist is IL-10, LL-37, or thymosin β4.

41. The method of claim 39, wherein the inflammatory cytokine antagonist is an antibody.

42. The method of claim 41, wherein the antibody is an anti-TNF-α antibody, an anti-IL-6 antibody, or an anti-IL-10 antibody.

43. The method of claim 39, wherein the anti-microbial factor is LL-37 or thymosin β4.

44. The method of claim 35, wherein the stem cell recruitment factor is TGF-β3, stromal cell-derived factor (SDF)-1-α, or thymosin β4.

45. The method of claim 35, wherein the extracellular matrix factor is collagen, laminin, or fibronectin.

46. The method of claim 35, wherein the cytokine or growth factor is selected from the group consisting of interleukins (ILs), epidermal growth factor (EGF), fibroblast growth factors (FGFs), platelet-derived growth factors (PDGFs), keratinocyte growth factor (KGF), bone morphogenetic proteins (BMPs), and colony stimulating factors (CSFs).

47. The method of claim 46, wherein the interleukin is IL-2 or IL-10.

48. The method of claim 46, wherein the FGF is FGF-1, FGF-2, FGF-7, or FGF-10.

49. The method of claim 46, wherein the BMP is selected from the group consisting of BMP-2, BMP-4, BMP-6, and BMP-7.

50. The method of claim 46, wherein the CSF is GM-CSF.

51. The method of claim 35, wherein the clotting factor is selected from factor I, factor II, CD142, factor V, factor VII, factor VIII, factor IX, factor X, factor XI, factor XII, factor XIII, von Willebrand factor, prekallikrein, high-molecular weight kininogen (HMWK), fibronectin, antithrombin III, heparin cofactor II, protein C, protein S, protein Z, plasminogen, tissue plasminogen activator (tPA), and urokinase.

52. The method of claim 35, wherein the angiogenic factor is a vascular endothelial growth factor (VEGF) or an angiopoetin.

53. The method of claim 52, wherein the VEGF is selected from the group consisting of VEGF-A, VEGF-B, VEGF-C, VEGF-D, VEGF-E, and placental growth factor (PIGF).

54. The method of claim 52, wherein the angiopoietin is ANGPT1 or ANGPT2.

55. The method of any one of claims 34-54, wherein the HUCPVC synthesizes and secretes the wound healing agent.

56. The method of any one of claims 34-55, wherein the HUCPVC has been genetically modified to express two or more wound-healing agents.

57. The method of any one of claims 34-56, wherein the HUCPVC has been genetically modified by viral transduction, transfection, dendrimers, gene editing, or a combination thereof.

58. The method of claim 57, wherein the viral transduction comprises adenoviral transduction, AAV transduction, or retroviral transduction.

59. The method of claim 58, wherein the retroviral transduction is lentiviral transduction.

60. The method of claim 57, wherein the transfection comprises naked nucleic acid transfection, electroporation, gene gun transfection, lipoplex transfection, or polyplex transfection.

61. The method of claim 57, wherein the gene editing comprises CRISPR-Cas gene editing, transcription activator-like effector based nuclease (TALEN) gene editing, zinc-finger nuclease (ZFN) gene editing, or meganuclease gene editing.

62. The method of any one of claims 34-61, wherein the wound healing agent is endogenous to the HUCPVC.

63. The method of any one of claims 34-61, wherein the wound healing agent is not endogenous to the HUCPVC.

64. The method of any one of claims 34-63, wherein the HUCPVCs have a 3G5+, CD45−, CD44+ phenotype.

65. The method of any one of claims 34-64, wherein the wound healing agent is a wild-type wound healing agent or a variant wound healing agent.

66. The method of claim 65, wherein the variant wound healing agent is a fusion protein.

67. The method of claim 66, wherein the fusion protein comprises a fusion partner selected from a targeting moiety and a detectable moiety.

68. The method of claim 67, wherein the targeting moiety comprises a CAR peptide (CARSKNKDC, SEQ ID NO: 1).

69. The method of claim 67, wherein the detectable moiety is an epitope tag or a fluorescent protein.

70. The method of any one of claims 33-69, wherein the subject is a vertebrate.

71. The method of claim 70, wherein the vertebrate is a mammal.

72. The method of claim 71, wherein the mammal is a human.

73. The method of any one of claims 33-72, wherein the genetically modified HUCPVC is allogeneic or xenogeneic to the subject.

74. The method of any one of claims 33-73, wherein the method comprises administering a single dose of the pharmaceutical composition.

75. The method of any one of claims 33-73, wherein the method comprises administering multiple doses of the pharmaceutical composition.

76. The method of any one of claims 33-75, wherein the genetically modified HUCPVC persists in the subject for greater than one week.

77. The method of claim 76, wherein the genetically modified HUCPVC persists in the subject for greater than one month.

78. The method of claim 77, wherein the genetically modified HUCPVC persists in the subject for greater than two months.

79. The method of any one of claims 33-78, wherein the pharmaceutical composition is administered to the subject intravenously, intramuscularly, subcutaneously, orally, by inhalation, parenterally, intraperitoneally, intraarterially, transdermally, sublingually, nasally, transbuccally, liposomally, adiposally, opthalmically, intraocularly, intrathecally, topically, or locally.

80. The method of any one of claims 33-79, wherein the HUCPVC evades immune recognition in the subject.

81. The method of any one of claims 33-80, wherein the subject is administered between 10¹ and 10¹³ HUCPVCs per dose.

82. The method of claim 81, wherein the subject is administered between 10³ and 10⁸ HUCPVCs per dose.

83. The method of any one of claims 33-82, further comprising administering at least one mesenchymal stem cell (MSC), wherein the MSC is not a HUCPVC.

84. The method of claim 83, wherein the MSC has been genetically modified to express a wound healing agent.

85. The method of claim 84, wherein the wound healing agent is selected from an anti-fibrotic factor, an anti-inflammatory factor, a stem cell recruitment factor, an extracellular matrix factor, a cytokine or growth factor, a clotting factor, and an angiogenic factor.

86. The method of any one of claims 83-85, wherein the MSC is isolated from bone marrow, umbilical cord blood, embryonic yolk sac, placenta, skin, or blood.

87. The method of any one of claims 33-86, further comprising administering one or more additional therapeutic agents to the subject.

88. The method of claim 87, wherein the one or more additional therapeutic agents enhances or prolongs the therapeutic benefit of the HUCPVC treatment.

89. The method of claim 87 or 88, wherein the one or more additional therapeutic agents is selected from the group consisting of an anti-microbial agent, an anti-inflammatory compound, a cytokine or growth factor, an analgesic, or an immunosuppressant.

90. The method of any one of claims 33-89, wherein the wound is an open wound, a closed wound, a chronic wound, or a burn.

91. The method of claim 90, wherein the open wound is selected from the group consisting of an incision, a laceration, an abrasion, an avulsion, a puncture wound, a penetration wound, and a gunshot wound.

92. The method of claim 90, wherein the closed wound is a hematoma or a crush injury.

93. The method of claim 90, wherein the chronic wound is a venous ulcer, a diabetic ulcer, or a pressure ulcer.

94. The method of claim 93, wherein the diabetic ulcer is a diabetic foot ulcer.

95. A method for producing a genetically modified HUCPVC, the method comprising introducing a nucleic acid encoding a wound healing agent into a HUCPVC, thereby producing a genetically modified HUCPVC expressing a wound healing agent.

96. The method of claim 95, wherein the wound healing agent is selected from the group consisting of an anti-fibrotic factor, an anti-inflammatory factor, a stem cell recruitment factor, an extracellular matrix factor, a cytokine or growth factor, a clotting factor, and an angiogenic factor.

97. The method of claim 96, wherein the anti-fibrotic factor is a TGF-β antagonist.

98. The method of claim 97, wherein the TGF-β antagonist is an anti-TGF-β antibody or a non-antibody TGF-β antagonist.

99. The method of claim 98, wherein the non-antibody TGF-β antagonist is decorin.

100. The method of claim 96, wherein the anti-inflammatory factor is an inflammatory cytokine antagonist or an anti-microbial factor.

101. The method of claim 100, wherein the inflammatory cytokine antagonist is IL-10, LL-37, or thymosin β4.

102. The method of claim 100, wherein the inflammatory cytokine antagonist is an antibody.

103. The method of claim 102, wherein the antibody is an anti-TNF-α antibody, an anti-IL-6 antibody, or an anti-IL-10 antibody.

104. The method of claim 100, wherein the anti-microbial factor is LL-37 or thymosin β4.

105. The method of claim 96, wherein the stem cell recruitment factor is TGF-β3, stromal cell-derived factor (SDF)-1-α, or thymosin β4.

106. The method of claim 96, wherein the extracellular matrix factor is collagen, laminin, or fibronectin.

107. The method of claim 96, wherein the cytokine or growth factor is selected from the group consisting of interleukins (ILs), epidermal growth factor (EGF), fibroblast growth factors (FGFs), platelet-derived growth factors (PDGFs), keratinocyte growth factor (KGF), bone morphogenetic proteins (BMPs), and colony stimulating factors (CSFs).

108. The method of claim 107, wherein the interleukin is IL-2 or IL-10.

109. The method of claim 107, wherein the FGF is FGF-1, FGF-2, FGF-7, or FGF-10.

110. The method of claim 107, wherein the BMP is selected from the group consisting of BMP-2, BMP-4, BMP-6, and BMP-7.

111. The method of claim 107, wherein the CSF is GM-CSF.

112. The method of claim 96, wherein the clotting factor is selected from factor I, factor II, CD142, factor V, factor VII, factor VIII, factor IX, factor X, factor XI, factor XII, factor XIII, von Willebrand factor, prekallikrein, high-molecular weight kininogen (HMWK), fibronectin, antithrombin III, heparin cofactor II, protein C, protein S, protein Z, plasminogen, tissue plasminogen activator (tPA), and urokinase.

113. The method of claim 96, wherein the angiogenic factor is a vascular endothelial growth factor (VEGF) or an angiopoetin.

114. The method of claim 113, wherein the VEGF is selected from the group consisting of VEGF-A, VEGF-B, VEGF-C, VEGF-D, VEGF-E, and placental growth factor (PIGF).

115. The method of claim 113, wherein the angiopoietin is ANGPT1 or ANGPT2.

116. The method of any one of claims 95-115, wherein the HUCPVC synthesizes and secretes the wound healing agent.

117. The method of any one of claims 95-116, wherein the HUCPVC is genetically modified to express two or more wound-healing agents.

118. The method of any one of claims 95-117, wherein the nucleic acid is introduced into the HUCPVC by viral transduction, transfection, dendrimers, gene editing, or a combination thereof.

119. The method of claim 118, wherein the viral transduction comprises adenoviral transduction, AAV transduction, or retroviral transduction.

120. The method of claim 119, wherein the retroviral transduction is lentiviral transduction.

121. The method of claim 118, wherein the transfection comprises naked nucleic acid transfection, electroporation, gene gun transfection, lipoplex transfection, or polyplex transfection.

122. The method of claim 118, wherein the gene editing comprises CRISPR-Cas gene editing, transcription activator-like effector based nuclease (TALEN) gene editing, zinc-finger nuclease (ZFN) gene editing, or meganuclease gene editing.

123. The method of any one of claims 95-122, wherein the wound healing agent is endogenous to the HUCPVC.

124. The method of any one of claims 95-122, wherein the wound healing agent is not endogenous to the HUCPVC.

125. The method of any one of claims 95-124, wherein the HUCPVCs have a 3G5+, CD45−, CD44+ phenotype.

126. The method of any one of claims 95-125, wherein the wound healing agent is a wild-type wound healing agent or a variant wound healing agent.

127. The method of claim 126, wherein the variant wound healing agent is a fusion protein.

128. The method of claim 127, wherein the fusion protein comprises a fusion partner selected from a targeting moiety and a detectable moiety.

129. The method of claim 128, wherein the targeting moiety comprises a CAR peptide (CARSKNKDC, SEQ ID NO: 1).

130. The method of claim 128, wherein the detectable moiety is an epitope tag or a fluorescent protein.

131. A method of treating a wound comprising administering a therapeutically effective amount of a pharmaceutical composition comprising the soluble fraction of medium conditioned by a HUCPVC, wherein the HUCPVC has been grown for one or more passages under substantially serum-free conditions.

132. The method of claim 131, wherein the composition comprises one or more additional soluble factors produced by the HUCPVC.

133. The method of claim 132, wherein the one or more soluble factors are paracrine factors.

134. The method of any one of claims 131-133, wherein the subject is a vertebrate.

135. The method of claim 134, wherein the vertebrate is a mammal.

136. The method of claim 135, wherein the mammal is a human.

137. The method of any one of claims 131-136, wherein the method comprises administering a single dose of the pharmaceutical composition.

138. The method of any one of claims 131-137, wherein the method comprises administering multiple doses of the pharmaceutical composition.

139. The method of any one of claims 131-138, wherein the pharmaceutical composition is administered to the subject intravenously, intramuscularly, subcutaneously, orally, by inhalation, parenterally, intraperitoneally, intraarterially, transdermally, sublingually, nasally, transbuccally, liposomally, adiposally, opthalmically, intraocularly, intrathecally, topically, or locally.

140. The method of any one of claims 131-139, further comprising administering at least one MSC or HUCPVC.

141. The method of claim 140, wherein the MSC or HUCPVC has been genetically modified to express a wound healing agent.

142. The method of claim 141, wherein the wound healing agent is selected from an anti-fibrotic factor, an anti-inflammatory factor, a stem cell recruitment factor, an extracellular matrix factor, a cytokine or growth factor, a clotting factor, and an angiogenic factor.

143. The method of any one of claims 131-142, wherein the MSC is isolated from bone marrow, umbilical cord blood, embryonic yolk sac, placenta, skin, or blood.

144. The method of any one of claims 131-143, further comprising administering one or more additional therapeutic agents to the subject.

145. The method of claim 144, wherein the one or more additional therapeutic agents enhances or prolongs the therapeutic benefit of the HUCPVC treatment.

146. The method of claim 144 or 145, wherein the one or more additional therapeutic agents is selected from the group consisting of an anti-microbial agent, an anti-inflammatory compound, a cytokine or growth factor, an analgesic, or an immunosuppressant.

147. The method of any one of claims 131-146, wherein the wound is an open wound, a closed wound, a chronic wound, or a burn.

148. The method of claim 147, wherein the open wound is selected from the group consisting of an incision, a laceration, an abrasion, an avulsion, a puncture wound, a penetration wound, and a gunshot wound.

149. The method of claim 147, wherein the closed wound is a hematoma or a crush injury.

150. The method of claim 147, wherein the chronic wound is a venous ulcer, a diabetic ulcer, or a pressure ulcer.

151. The method of claim 150, wherein the diabetic ulcer is a diabetic foot ulcer.

152. The method of claim 141, wherein the wound healing agent is decorin.
